(19) United States
(12) Patent Application Publication
(10) Pub. No.: US 2024/0382767 A1
(43) Pub. Date: Nov. 21, 2024

(54) WIRELESS COMMUNICATION AND POWER HARVESTING FOR IMPLANTABLE DEVICES

(71) Applicant: XII Medical, Inc., Union City, CA (US)

(72) Inventors: Rabih Nassif, Santa Ana, CA (US); Paul F. Wittibschlager, Macedonia, OH (US); Paramjit Singh Banwait, Fremont, CA (US); Steven M. Young, Ann Arbor, MI (US); Anthony V. Caparso, North Ridgeville, OH (US)

(21) Appl. No.: 18/664,984

(22) Filed: May 15, 2024

Related U.S. Application Data
(60) Provisional application No. 63/503,214, filed on May 19, 2023.

(51) Int. Cl. A61N 1/372; H01Q 1/22; H04B 5/70

(52) U.S. Cl. CPC A61N 1/37229; H01Q 1/2291; H04B 5/70

(57) ABSTRACT

According to some embodiments, the present technology includes an implantable device comprising a flexible circuit including a substrate carrying a first antenna (e.g., a power harvesting antenna) configured to operate at a first frequency and a second antenna (e.g., a Bluetooth antenna) configured to operate at a second frequency different from the first frequency. The first antenna can be configured to induce a current in response to being disposed in an alternating electromagnetic field, and the second antenna can be configured to radiate radiofrequency energy to transmit data to an external device.

### CROSS-REFERENCE TO RELATED APPLICATIONS(S)

The present technology claims the benefit of priority to U.S. Provisional Patent Application No. 63/503,214, filed May 19, 2023, titled WIRELESS COMMUNICATION AND POWER HARVESTING FOR IMPLANTABLE DEVICES, which is incorporated by reference herein in its entirety.

The present application is also related to U.S. patent application Ser. No. 18/437,135, filed Feb. 8, 2024, titled WIRELESS POWER TRANSFER, and U.S. Patent Application No. 63,573,726, filed Apr. 3, 2024, titled NEUROMODULATION DEVICES AND ASSOCIATED SYSTEMS AND METHODS, each of which is incorporated by reference herein in its entirety.

### TECHNICAL FIELD

The present technology relates to wireless communication devices, systems, and methods. Various embodiments of the present technology relate to devices, systems, and methods for enabling wireless communication with implantable neuromodulation devices.

### BACKGROUND

Sleep disordered breathing (SDB), such as upper airway sleep disorders (UASDs), is a condition that occurs that diminishes sleep time and sleep quality, resulting in patients exhibiting symptoms that include daytime sleepiness, tiredness, and lack of concentration. Obstructive sleep apnea (OSA), the most common type of SDB, affects one in five adults in the United States. One in 15 adults has moderate to severe OSA and requires treatment. Untreated OSA results in reduced quality of life measures and increased risk of disease, including hypertension, stroke, heart disease, and others.

OSA is characterized by the complete obstruction of the airway, causing breathing to cease completely (apnea) or partially (hypopnea). During sleep, the tongue muscles relax. In this relaxed state, the tongue may lack sufficient muscle tone to prevent the tongue from changing its normal tonic shape and position. When the base of the tongue and/or soft tissue of the upper airway collapse, the upper airway channel is blocked, causing an apnea event. Blockage of the upper airway prevents air from flowing into the lungs, thereby decreasing the patient's blood oxygen level, which in turn increases blood pressure and heart dilation. This causes a reflexive forced opening of the upper airway channel until normal patency is regained, followed by normal respiration until the next apneic event. These reflexive forced openings briefly arouse the patient from sleep.

Current treatment options range from drug intervention, non-invasive approaches, to more invasive surgical procedures. In many of these instances, patient acceptance and therapy compliance are well below desired levels, rendering the current solutions ineffective as a long-term solution. Continuous positive airway pressure (CPAP), for example, is a standard treatment for OSA. While CPAP is non-invasive and highly effective, it is not well tolerated by all patients and has several side effects. Patient compliance and/or tolerance for CPAP is often reported to be between 40% and 60%. Surgical treatment options for OSA, such as anterior tongue muscle repositioning, orthognathic bimaxillary advancement, uvula-palatalpharyngoplasty, and tracheostomy are available too. However, these procedures tend to be highly invasive, irreversible, and have poor and/or inconsistent efficacy. Even the more effective surgical procedures are undesirable because they usually require multiple invasive and irreversible operations, they may alter a patient's appearance (e.g., maxillo-mandibular advancement), and/or they may be socially stigmatic (e.g., tracheostomy) and have extensive morbidity.

### SUMMARY

The subject technology is illustrated, for example, according to various aspects described below, including with reference to FIGS. 1A-15B. Various examples of aspects of the subject technology are described as numbered Examples (1, 2, 3, etc.) for convenience. These are provided as examples and do not limit the subject technology.

Example 1. An implantable device comprising:
a flexible circuit including a substrate carrying:
a power harvesting antenna configured to operate at a first frequency, the power harvesting antenna being configured to induce a current in response to being disposed in an alternating electromagnetic field; and
a Bluetooth antenna configured to operate at a second frequency different than the first frequency, the Bluetooth antenna being configured to radiate radiofrequency energy to transmit data to an external device.

Example 2. The device of Example 1, wherein the power harvesting antenna is configured to operate between about 100 kHz and about 900 MHz and the Bluetooth antenna is configured to operate between about 2.4 GHz and about 2.483 GHz.

Example 3. The device of Example 1 or 2, wherein the substrate comprises a first broad surface and a second broad surface opposite the first broad surface along a thickness of the substrate.

Example 4. The device of any one of Examples 1-3, wherein each of the first and second broad surfaces is substantially ovular.

Example 5. The device of any one of Examples 1-4, wherein the thickness of the substrate is between about 0.3 mm and about 0.5 mm.

Example 6. The device of any one of Examples 1-5, wherein the substrate comprises multiple layers.

Example 7. The device of Example 6, wherein the Bluetooth antenna is located on a different layer of the multiple layers of the substrate than the power harvesting antenna.

Example 8. The device of Example 6 or 7, wherein the power harvesting antenna is located on at least two of the multiple layers of the substrate.

Example 9. The device of any one of Examples 1-8, wherein the power harvesting antenna comprises a plurality of turns.

Example 10. The device of Example 9, wherein the Bluetooth antenna is located radially within an innermost turn of the plurality of turns.

Example 11. The device of Example 9 or 10, wherein the Bluetooth antenna is radially separated from the innermost turn of the power harvesting antenna by at least about 0.5 mm.

Example 12. The device of any one of Examples 9-11, wherein the flexible circuit further comprises power harvesting circuitry and a power harvesting circuitry connection connecting an outermost turn of the plurality of turns to the power harvesting circuitry, wherein the power harvesting circuitry trace extends orthogonally to a superimposed portion of the Bluetooth antenna.

Example 13. The device of any one of Examples 9-12, wherein the Bluetooth antenna comprises a Bluetooth antenna path extending from a first end to a second end, and wherein the first end is radially within an innermost turn of the plurality of turns of the power harvesting antenna and the second end is radially outside of an outermost turn of the plurality of turns of the power harvesting antenna, such that the Bluetooth antenna path extends across the plurality of turns.

Example 14. The device of Example 13, wherein the Bluetooth antenna path extends orthogonally across at least a portion of the plurality of turns of the power harvesting antenna.

Example 15. The device of any one of Examples 1-14, wherein the Bluetooth antenna comprises a dipole.

Example 16. The device of any one of Examples 1-15, wherein the power harvesting antenna and the Bluetooth antenna are physically separated such that an amplitude of a signal generated by flow of induced current within the power harvesting antenna at the second frequency is less than a noise floor of the Bluetooth antenna.

Example 17. The device of any one of Examples 1-16, wherein the flexible circuit comprises a ground plate electrically coupled to the power harvesting antenna and electrically isolated from the Bluetooth antenna.

Example 18. The device of any one of Examples 1-17, wherein the flexible circuit comprises a matching circuit electrically coupled to the Bluetooth antenna.

Example 19. The device of Example 18, wherein the matching circuit is configured to prevent or limit transmission of a component of the alternating electromagnetic field at the first frequency through the matching circuit.

Example 20. The device of Example 18 or 19, wherein the matching circuit comprises a capacitor having an impedance of at least about 10K ohms at the first frequency of the power harvesting antenna.

Example 21. The device of any one of Examples 1-20, wherein the second frequency is within a predetermined frequency range defined by a maximum frequency and a minimum frequency.

Example 22. The device of Example 21, wherein a return loss of the Bluetooth antenna at any given frequency within the predetermined frequency range is at least −10 dB.

Example 23. The device of Example 21 or 22, wherein the predetermined frequency range is discretized into a plurality of frequency bands, and wherein a return loss of the Bluetooth antenna within each frequency band of the plurality of frequency bands is within 10% of the return loss of the Bluetooth antenna within every other frequency band of the plurality of frequency bands.

Example 24. The device of any one of Examples 21-23, wherein a return loss of the Bluetooth antenna is maximized at a center frequency of the predetermined frequency range, the center frequency being about halfway between the minimum frequency and the maximum frequency.

Example 25. The device of any one of Examples 1-24, wherein the Bluetooth antenna is configured to have a first impedance once the implantable device is positioned within a patient, and wherein the flexible circuit comprises:
a Bluetooth module having a second impedance different than the first impedance, the Bluetooth module being configured to transmit a radiofrequency signal to the Bluetooth antenna to cause the Bluetooth antenna to radiate the radiofrequency energy; and
a matching circuit electrically coupled to and positioned between the Bluetooth module and the Bluetooth antenna, the matching circuit having a third impedance, the third impedance being equal to a difference between the second impedance and the first impedance.

Example 26. The device of Example 25, wherein the first impedance of the Bluetooth antenna is inductive.

Example 27. The device of Example 25 or 26, wherein the second impedance of the Bluetooth module is about 50 Ohms.

Example 28. The device of any one of Examples 25-27, wherein the matching circuit is a reactive matching circuit.

Example 29. The device of Example 28, wherein the matching circuit includes three reactive electronic components, five reactive electronic components, or seven reactive electronic components.

Example 30. The device of Example 28 or 29, wherein the matching circuit includes a first series capacitor, a second series capacitor, and a shunt capacitor therebetween.

Example 31. The device of Example 30, wherein each of the first series capacitor, the second series capacitor, and the shunt capacitor has have a self-resonant frequency of at least about 4 GHz.

Example 32. The device of any one of Examples 25-31, wherein the matching circuit includes a thin film capacitor.

Example 33. The device of any one of Examples 1-32, wherein the flexible circuit is encapsulated in a coating comprising an epoxy.

Example 34. The device of Example 33, wherein the coating has a maximum thickness of between about 1.5 mm and about 2 mm.

Example 35. A system comprising:
an external device configured to be positioned external to a body of a patient, the external device comprising an external Bluetooth antenna; and
an implantable device comprising a flexible circuit including a substrate having a first broad side and a second broad side opposite the first broad side along a thickness of the substrate, the substrate carrying an implantable Bluetooth antenna located closer to the first broad side than the second broad side along the thickness of the substrate, wherein the implantable device is configured to be positioned within the body of the patient such that the first broad side is located closer to the external Bluetooth antenna than the second broad side.

Example 36. The system of Example 35, wherein the implantable Bluetooth antenna is configured to radiate radiofrequency energy through the body of the patient to the external Bluetooth antenna.

Example 37. The system of Example 35 or 36, wherein the implantable Bluetooth antenna is configured to radiate little to no radiofrequency energy through air of an environment external of the patient to the external Bluetooth antenna.

Example 38. The system of any one of Examples 35-37, wherein the implantable device is configured to be positioned within an under-chin region of the patient and the external device is configured to be positioned proximate a head of the patient.

Example 39. The system of any one of Examples 35-38, wherein the external device is configured to be positioned proximate a top of the head of the patient.

Example 40. The system of any one of Examples 35-39, wherein the implantable device is configured to be positioned within an under-chin region of the patient with the first broad surface cranial to the second broad surface.

Example 41. The system of any one of Examples 35-40, wherein the implantable device is configured to be positioned within an under-chin region of the body of the patient with the first broad surface proximate to a mylohyoid muscle of the patient and the second broad surface spaced apart from the mylohyoid muscle by the thickness of the substrate.

Example 42. A method comprising:
positioning an external device proximate to a body of a patient, the external device comprising an external Bluetooth antenna; and
implanting an implantable device within the body of the patient, the implantable device comprising a flexible circuit including a substrate having a first broad side and a second broad side opposite the first broad side along a thickness of the substrate, the substrate carrying an implantable Bluetooth antenna located closer to the first broad side than the second broad side along the thickness of the substrate,
wherein implanting the implantable device comprises positioning the implantable device within the body of the patient such that the first broad side is located closer to the external Bluetooth antenna than the second broad side.

Example 43. The method of Example 42, wherein the implantable Bluetooth antenna is configured to radiate radiofrequency energy through the body of the patient to the external Bluetooth antenna.

Example 44. The method of Example 42 or 43, wherein the implantable Bluetooth antenna is configured to radiate little to no radiofrequency energy through air of an environment external of the patient to the external Bluetooth antenna.

Example 45. The method of any one of Examples 42-44, wherein implanting the implantable device within the body of the patient comprises positioning the implantable device within an under-chin region of the patient.

Example 46. The method of Example 45, wherein positioning the implantable device within the under-chin region of the patient comprises positioning the first broad surface cranial to the second broad surface.

Example 47. The method of Example 45 or 46, wherein positioning the implantable device within the under-chin region of the patient comprises positioning the first broad surface proximate to a mylohyoid muscle of the patient with the second broad surface spaced apart from the mylohyoid muscle by the thickness of the substrate.

Example 48. The method of any one of Examples 42-47, wherein positioning the external device proximate to the body of the patient comprises positioning the external device proximate to a head of the patient.

Example 49. The method of any one of Examples 42-48, wherein positioning the external device proximate to the body of the patient comprises positioning the external device proximate to a top of a head of the patient.

Example 50. An implantable device comprising:
a flexible circuit including a substrate carrying:
a first antenna configured to operate at a first frequency, the first antenna being configured to induce a current in response to being disposed in an alternating electromagnetic field; and
a second antenna configured to operate at a second frequency different than the first frequency, the second antenna being configured to radiate radiofrequency energy to transmit data to an external device.

Example 51. A system comprising:
an external device configured to be positioned external to a body of a patient, the external device comprising an external antenna; and
an implantable device comprising a flexible circuit including a substrate having a first broad side and a second broad side opposite the first broad side along a thickness of the substrate, the substrate carrying an implantable antenna configured to wirelessly communicate with the external antenna,
wherein the implantable antenna is located closer to the first broad side than the second broad side along the thickness of the substrate, and
wherein the implantable device is configured to be positioned within the body of the patient such that the first broad side is located closer to the external antenna than the second broad side.

Example 52. A method comprising:
positioning an external device proximate to a body of a patient, the external device comprising an external antenna; and
implanting an implantable device within the body of the patient, the implantable device comprising a flexible circuit including a substrate having a first broad side and a second broad side opposite the first broad side along a thickness of the substrate, the substrate carrying an implantable antenna configured to wirelessly communicate with the external antenna, wherein the implantable antenna is located closer to the first broad side than the second broad side along the thickness of the substrate,
wherein implanting the implantable device comprises positioning the implantable device within the body of the patient such that the first broad side is located closer to the external antenna than the second broad side.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a fragmentary midline sagittal view of an upper airway of a human patient.

FIG. 1B is an illustration of the musculature and hypoglossal innervation of the human tongue.

FIG. 1C is a schematic superior view of a distal arborization of right and left hypoglossal nerves of a human patient. The hypoglossal nerves of FIG. 1C are shown as extending anteriorly from the bottom of the page to the top of the page (e.g., from the hyoid bone to the anterior mandible).

FIG. 2A is a schematic illustration of an example embodiment of a neuromodulation system configured in accordance with the present technology.

FIG. 2B is a perspective view of an example embodiment of a neuromodulation device configured in accordance with the present technology.

FIGS. 2C and 2D are top and side views, respectively, of the neuromodulation device of FIG. 2B.

FIGS. 3A-3F are various views of the neuromodulation device shown in FIGS. 2B-2D implanted in a human patient in accordance with the present technology.

FIGS. 4A and 4B are perspective views of an example embodiment of an external device in accordance with the present technology. FIG. 4C is a cutaway perspective view of the external device of FIGS. 4A and 4B, and FIG. 4D is a side cross-sectional view of the external device of FIGS. 4A and 4B.

FIG. 5A is a schematic illustration of an example embodiment of a flexible circuit including a power harvesting antenna and a communications antenna, configured in accordance with the present technology. FIG. 5B is a side partial cross-sectional view of the flexible circuit of FIG. 5A.

FIGS. 6A and 6B are schematic side and detailed views, respectively, of an example embodiment of an electronics package implanted in a patient, configured in accordance with the present technology.

FIG. 7 is a plot illustrating desired amplitude of a power harvesting antenna signal and/or its harmonics, relative to the operational range of a communications antenna, configured in accordance with the present technology.

FIG. 8A is a plot illustrating return loss of an example embodiment of a communications antenna arrangement with a matching circuit, configured in accordance with the present technology.

FIG. 8B is a plot illustrating return loss of various example embodiments of communications antenna arrangements with a matching circuit, configured in accordance with the present technology.

FIG. 9 is a schematic illustration of an example embodiment of a matching circuit configured in accordance with the present technology.

FIG. 10A is a schematic illustration of an example embodiment of a flexible circuit including a power harvesting antenna and a communications antenna, configured in accordance with the present technology. FIG. 10B is a side partial cross-sectional view of the flexible circuit of FIG. 10A.

FIG. 11A is a schematic illustration of an example embodiment of a flexible circuit including a power harvesting antenna and a communications antenna, configured in accordance with the present technology. FIG. 11B is a side partial cross-sectional view of the flexible circuit of FIG. 10A.

FIG. 12A is a schematic illustration of an example embodiment of a flexible circuit including a power harvesting antenna and a communications antenna, configured in accordance with the present technology. FIG. 12B is a side partial cross-sectional view of the flexible circuit of FIG. 10A.

FIG. 13A is a schematic illustration of an example embodiment of a flexible circuit including a communications antenna, configured in accordance with the present technology. FIG. 13B is a side partial cross-sectional view of a substrate including the flexible circuit of FIG. 13A.

FIG. 14A is a schematic illustration of an example embodiment of a flexible circuit including a power harvesting antenna and connections for a communications antenna, configured in accordance with the present technology. FIG. 14B is a schematic illustration of an example embodiment of a neuromodulation device including the flexible circuit of FIG. 14A and wires of a communications antenna extending along the neuromodulation device, configured in accordance with the present technology. FIG. 14C is a cross-sectional view of a portion of the neuromodulation device of FIG. 14B.

FIG. 15A is a cross-sectional view of a portion of an example embodiment of a lead body in a neuromodulation device including a wire for a communications antenna, configured in accordance with the present technology.

FIG. 15B is a cross-sectional view of a portion of an example embodiment of a lead body in a neuromodulation device including a wire for a communications antenna, configured in accordance with the present technology.

### DETAILED DESCRIPTION

The present disclosure relates to devices, systems, and methods for wirelessly powering implantable medical devices. For example, an external system of the present technology can comprise a control unit coupled to an external device comprising a carrier carrying an antenna configured to conduct electrical current such that the antenna generates an electromagnetic field. When the implantable device is positioned within the electromagnetic field generated by the antenna, current can be induced in an antenna of the implantable device that can be used to power one or more electronic components carried by the implantable device. In some embodiments, the external devices and systems disclosed herein are used to power a neuromodulation system, which can be used to provide a variety of electrical therapies, including neuromodulation therapies such as nerve and/or muscle stimulation. Stimulation can induce excitatory or inhibitory neural or muscular activity. Such therapies can be used at various suitable sites within a patient's anatomy. According to some embodiments, the neuromodulation systems of the present technology are configured to treat sleep disordered breathing (SDB), including obstructive sleep apnea (OSA) and/or mixed sleep apnea, via neuromodulation of the hypoglossal nerve (HGN).

For the purpose of contextualizing the structure and operation of the neuromodulation systems and devices disclosed herein, some of the relevant anatomy and physiology are first described below. The headings provided herein are for convenience only and do not interpret the scope or meaning of the claimed present technology. Embodiments under any one heading may be used in conjunction with embodiments under any other heading. For example, any of the neuromodulation systems and devices described in connection with Sections II and III can include any of the electronics packages described in connection with Sections V and/or can be used with any of the external systems and external devices described in connection with Section IV.

### I. Anatomy and Physiology

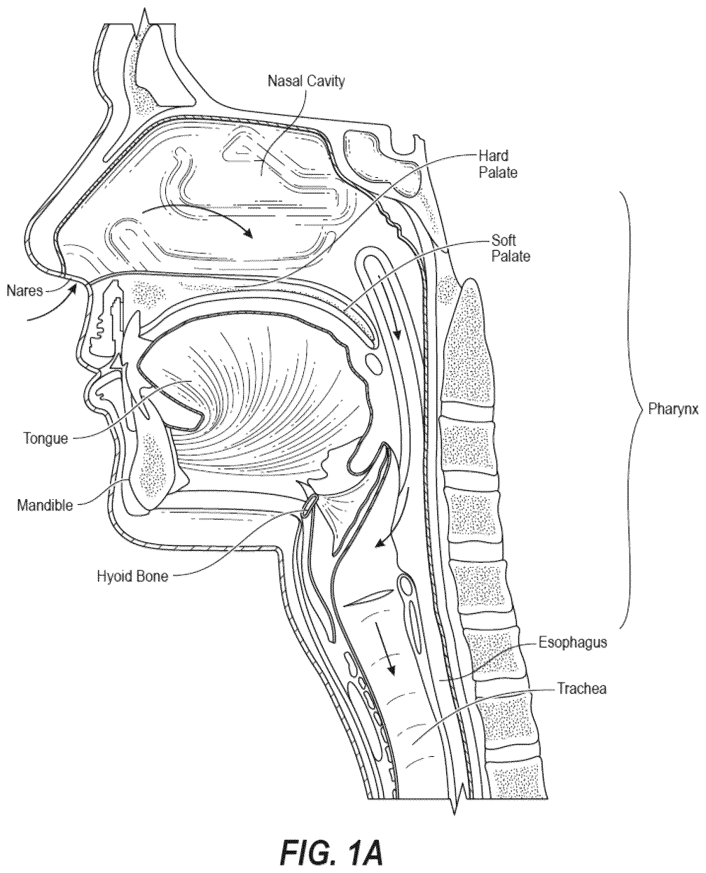

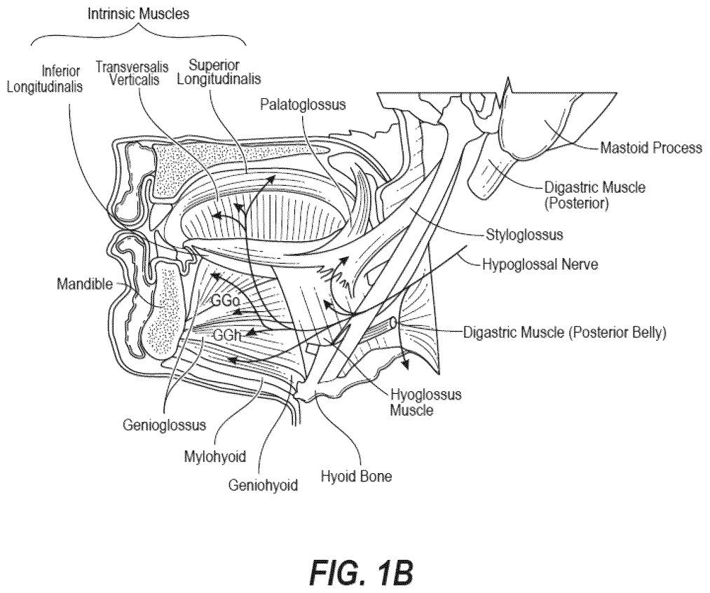

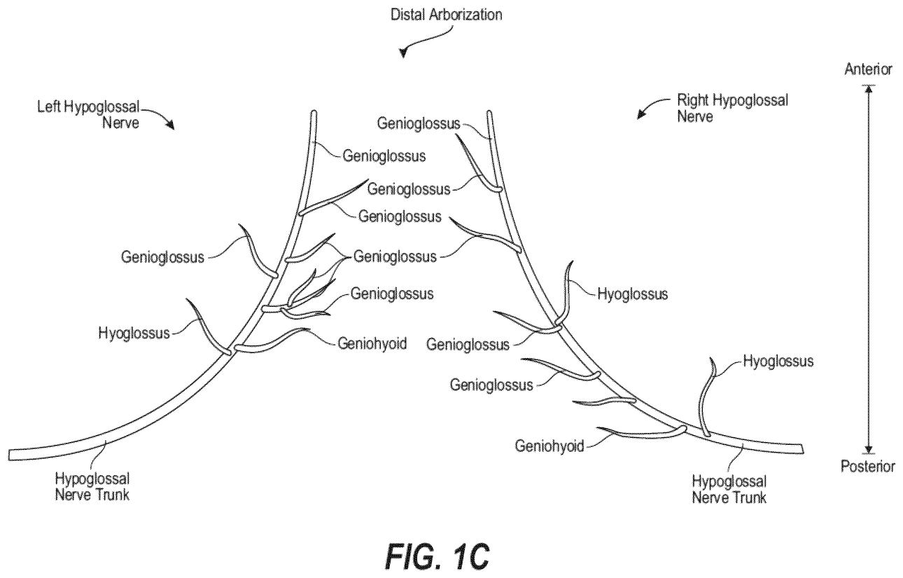

As previously mentioned, respiration in patients with SDB is frustrated due to obstruction, narrowing, and/or collapse of the upper airway during sleep. As shown in FIG. 1A, the upper airway comprises the nasal cavity, the oral cavity, the pharynx, and the larynx. Patency of the upper airway and resistance to airflow in the upper airway are controlled by a complex network of muscles under both voluntary and involuntary neuromuscular control. For example, the muscles of the tongue, the suprahyoid muscles (e.g., the geniohyoid, mylohyoid, stylohyoid, hyoglossus, and the anterior belly of the digastric muscle), and the muscles comprising the soft palate (e.g., palatal muscles) open, widen, and/or stabilize the upper airway during inspiration to counteract the negative airway pressure responsible for drawing air into the airway and the lungs.

With reference to FIG. 1B, the tongue comprises both intrinsic and extrinsic lingual muscles. Generally, activation of the intrinsic muscles changes the shape of the tongue while activation of the extrinsic muscles tends to move the position of the whole tongue. The extrinsic muscles originate at a bony attachment and insert within the tongue. They comprise the genioglossus muscle, the styloglossus muscle, the hyoglossus muscle, and the palatoglossus muscle. The intrinsic muscles both originate and insert within the tongue, and comprise the superior longitudinalis, the inferior longitudinalis, the transversalis, and the verticalis. In a patient who is awake, the brain supplies neural drive to these muscles through the HGN to maintain tongue shape and position, preventing the tongue from blocking the airway.

The lingual muscles are also functionally categorized as either retrusor or protrusor muscles and both intrinsic and extrinsic muscles fall into these categories. The retrusor muscles include the intrinsic superior and inferior longitudinalis muscles and the extrinsic hyoglossus and styloglossus muscles. The protrusor muscles include the intrinsic verticalis and transversalis muscles and the extrinsic genioglossus muscle. Contraction of the styloglossus muscle causes elevation of the tongue while depression of the tongue is the result of downward movements of hyoglossus and genioglossus muscles. Also labeled in FIG. 1B is the geniohyoid muscle, which is a suprahyoid muscle (not a tongue muscle) but still an important protrusor and pharyngeal dilator, and thus contributes to maintaining upper airway patency. It is believed that effective treatment of OSA requires stimulation of the protrusor muscles with minimal or no activation of the retrusor muscles. Thus, for neuromodulation therapy to be effective it is considered beneficial to localize stimulation to the protrusor muscles while avoiding activation of the retrusor muscles.

The largest of the tongue muscles, the genioglossus, comprises two morphological and functional compartments according to fiber distribution, action, and nerve supply. The first, the oblique compartment (GGo), includes vertical fibers that, when contracted, depress the tongue without substantially affecting pharyngeal patency. The second, the horizontal compartment (GGh), contains longitudinal fibers that, when activated, protrude the posterior part of the tongue and enlarge the pharyngeal opening. The GGo contains Type II muscle fibers that are quickly fatigued, whereas the GGh contains Type I muscle fibers that are slower to fatigue. Accordingly, it can be advantageous to stimulate the GGh with little or no stimulation of the GGo to effectively protrude the tongue while preventing or limiting fatigue of the tongue.

The suprahyoid muscles, which comprise the mylohyoid, the geniohyoid, the stylohyoid, and the digastric (only a portion of which is shown in FIG. 1B), extend between the mandible and the hyoid bone to form the floor of the mouth. The geniohyoid is situated inferior to the genioglossus muscle of the tongue and the mylohyoid is situated inferior to the geniohyoid. Contraction of the geniohyoid and tone of the sternohyoid (an infrahyoid muscle, not shown) cooperate to pull the hyoid bone anteriorly to open and/or widen the pharyngeal lumen and stabilize the anterior wall of the hypopharyngeal region. In contrast to the genioglossus and geniohyoid, which are considered tongue protrusors, the hyoglossus and styloglossus are considered tongue retrusors. Activation of the hyoglossus and styloglossus tends to retract the tongue posteriorly, which reduces the size of the pharyngeal opening, increases airway resistance, and frustrates respiration.

As previously mentioned, all of the extrinsic and intrinsic muscles of the tongue are innervated by the HGN, with the exception of the palatoglossus, which is innervated by the vagal nerve. There are two hypoglossal nerves in the body, one on the right side of the head and one on the left side. Each hypoglossal nerve originates at a hypoglossal nucleus in the medulla oblongata of the brainstem, exits the cranium via the hypoglossal canal, and passes inferiorly through the retrostyloid space (a portion of the lateral pharyngeal space) to the occipital artery. The hypoglossal nerve then curves and courses anteriorly to the muscles of the tongue, passing between the anterior edge of the hyoglossus muscle and the posterior edge of the mylohyoid muscle into the sublingual area where it splits into a distal arborization.

FIG. 1C is a schematic superior view of the distal arborization of the right and left hypoglossal nerves. Referring to FIGS. 1B and 1C together, the HGN comprises (1) portions of the distal arborization that innervate the styloglossus and the hyoglossus (tongue retrusor muscles) and (2) portions of the distal arborization that innervate the intrinsic muscles of the tongue, the genioglossus, and the geniohyoid (tongue protrusor muscles). Additionally, the portions of the distal arborization that innervate the tongue retrusor muscles tend to be located posterior of the portions of the distal arborization that innervate the tongue protrusor muscles.

A reduction in activity of the muscles responsible for airway maintenance can result in an increase in airway resistance and a myriad of downstream effects on a patient's respiration and health. Activity of the genioglossus muscle, for example, can decrease during sleep which, whether alone or in combination with other factors (e.g., airway length, airway diameter, soft tissue volume, premature wakening, etc.), can result in substantial airway resistance and/or airway collapse leading to sleep disordered breathing, such as OSA. It is believed that in order for neuromodulation therapy to be effective, it may be beneficial to largely confine stimulation of the HGN to the portions of the distal arborization that innervate protrusor muscles while avoiding or limiting stimulation of the portions of the distal arborization that activate the retrusor muscles.

### II. Neuromodulation Systems

Various embodiments of the present technology are directed to devices, systems, and methods for modulating neurological activity and/or control of one or more nerves associated with one or more muscles involved in airway maintenance. Such neuromodulation can increase activity in targeted muscles, for example the genioglossus and geniohyoid, to reduce a patient's airway resistance and improve the patient's respiration. Moreover, targeted modulation of specific portions of the distal arborization of the hypoglossal nerve can increase activity in tongue protrusor muscles without substantially increasing activity in tongue retrusor muscles to provide a highly efficacious treatment. Additionally or alternatively, targeted modulation of specific portions of the distal arborization of the hypoglossal nerve that innervate the GGh but not portions of the distal arborization of the hypoglossal nerve that innervate the GGo can be used to effectively protrude the tongue while preventing or limiting fatigue of the tongue.

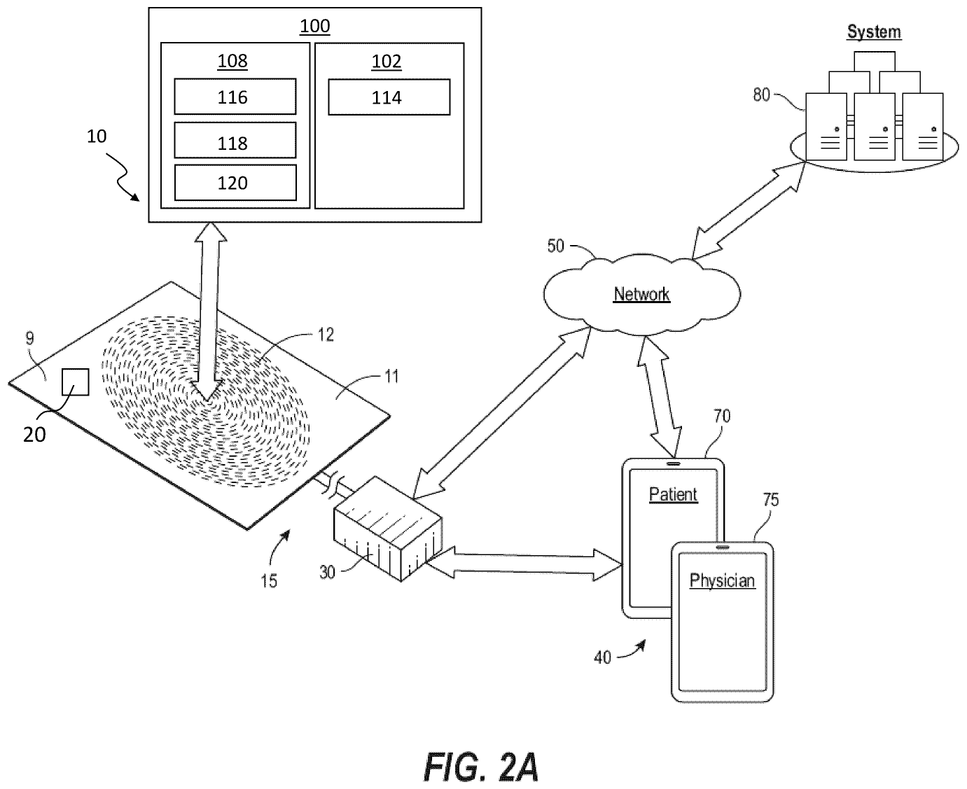

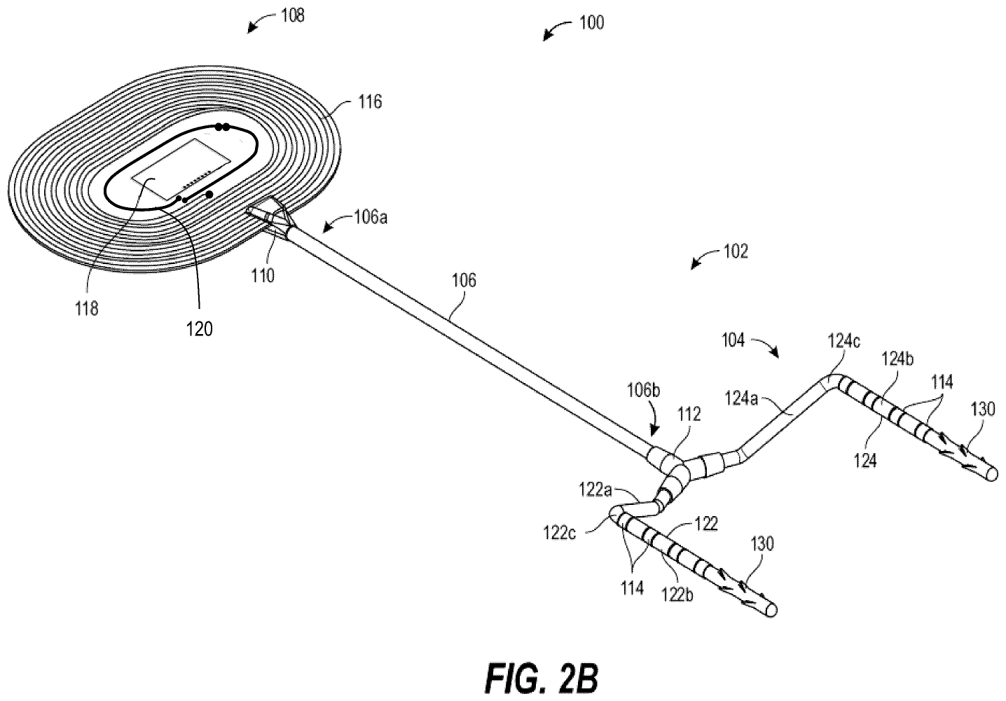

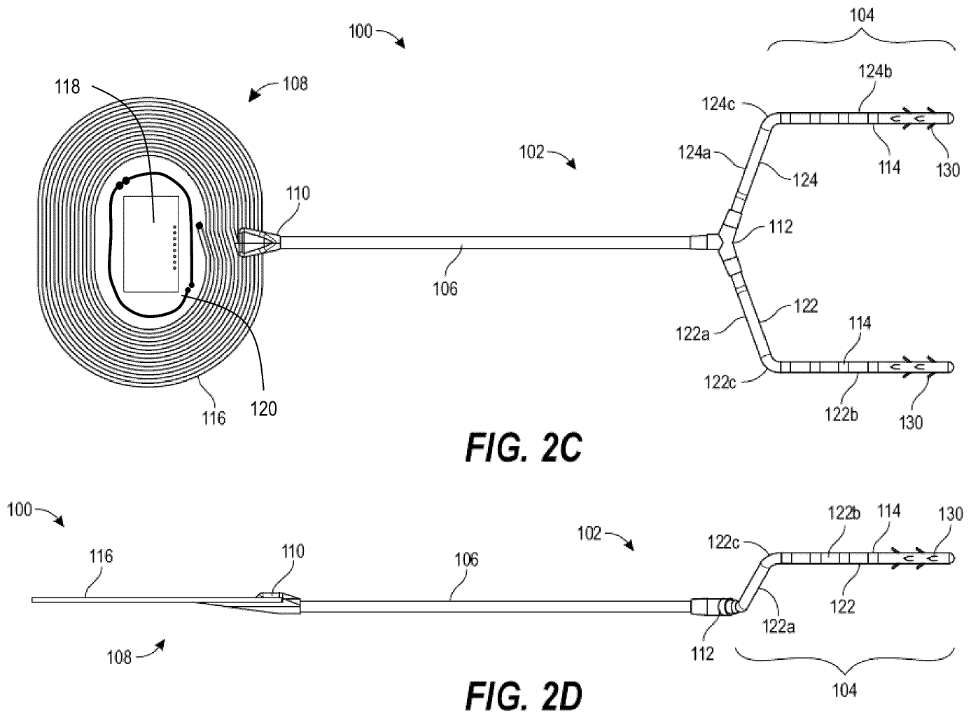

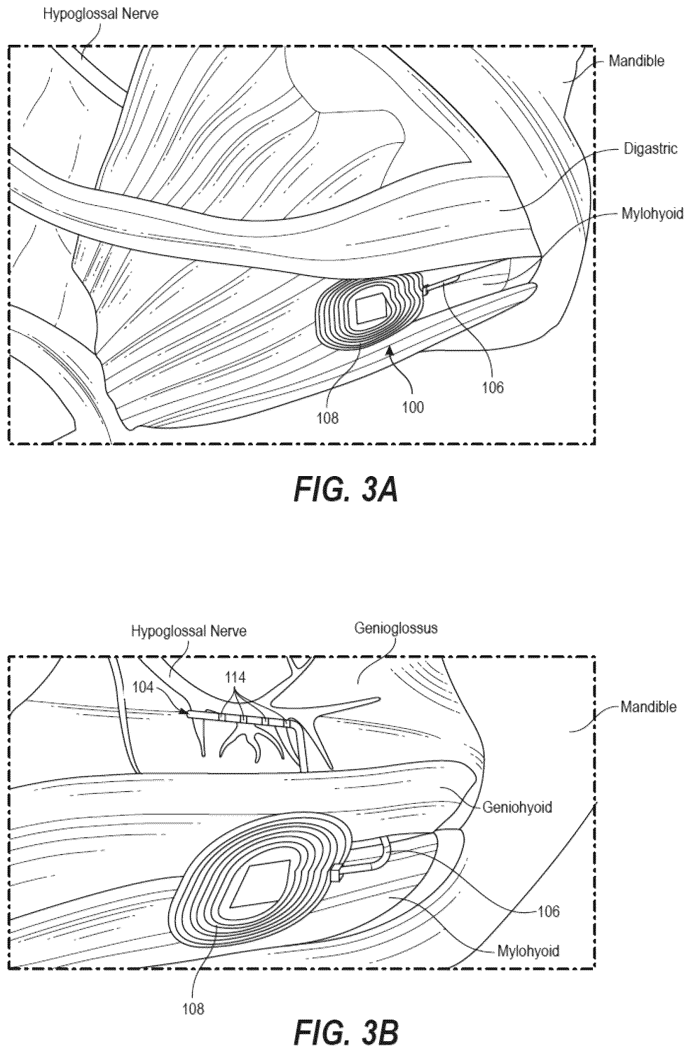

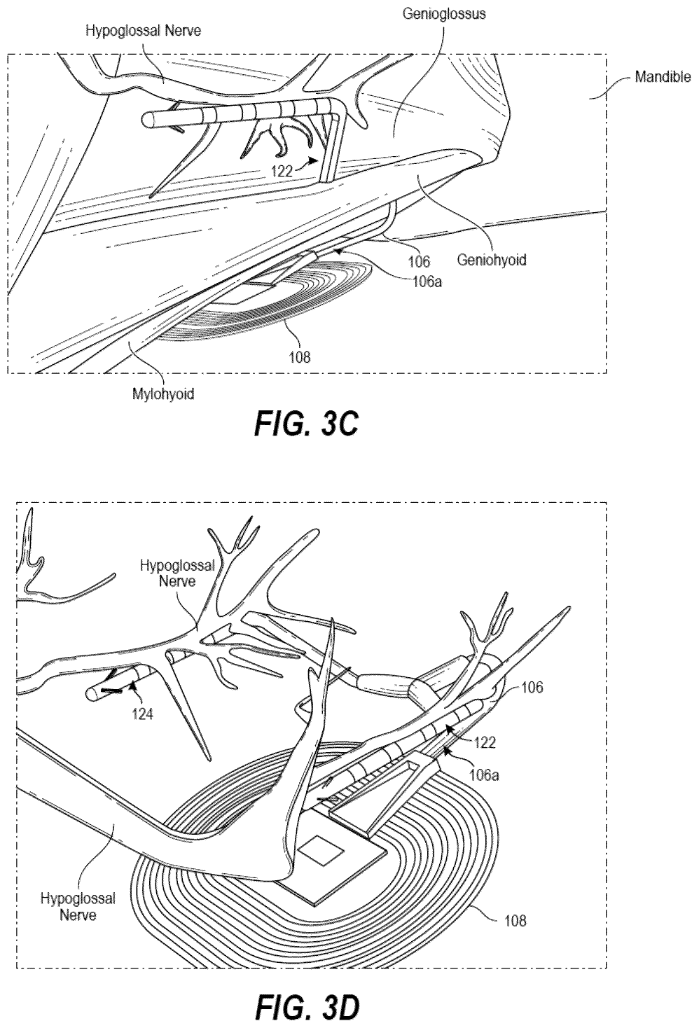

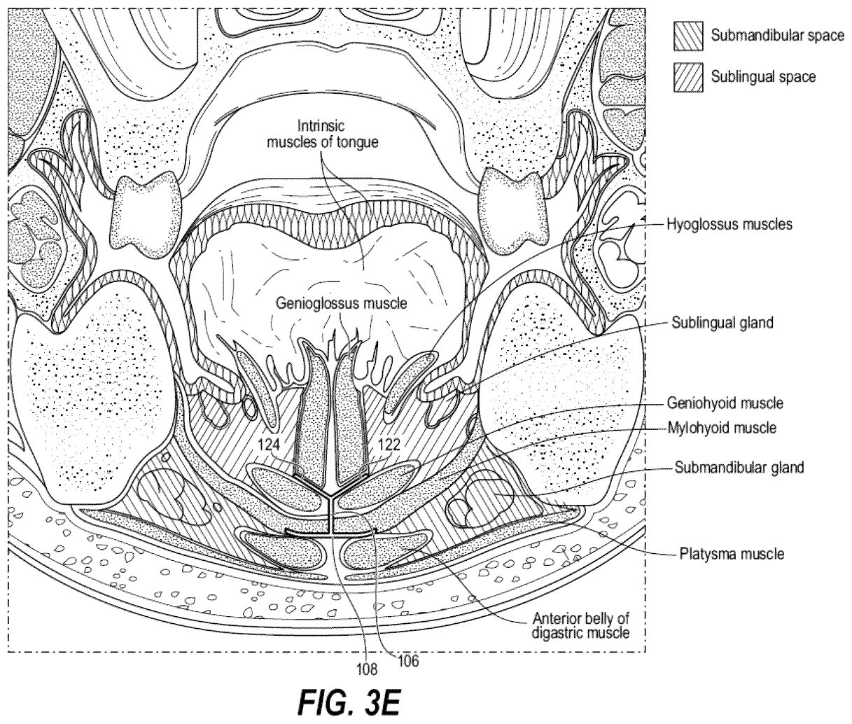

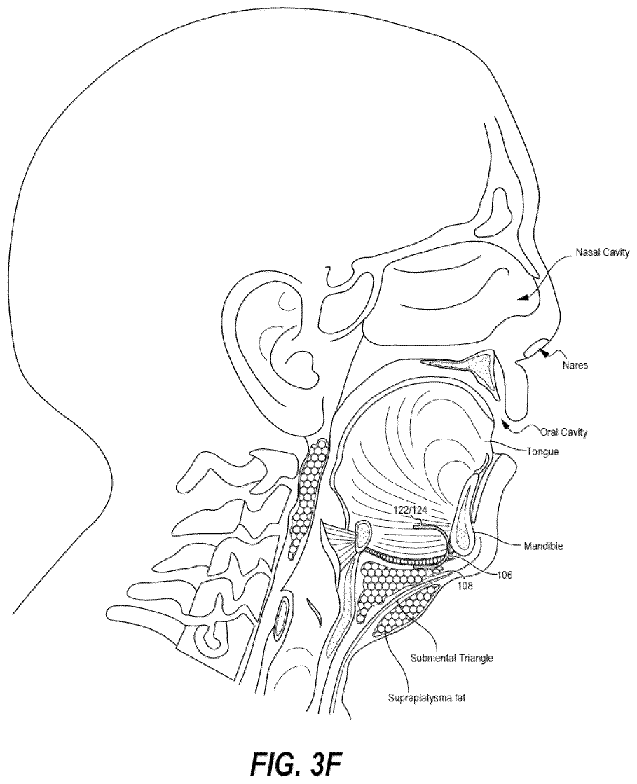

FIG. 2A shows a neuromodulation system 10 for treating SDB configured in accordance with the present technology. The system 10 can include an implantable neuromodulation device 100 and an external system 15 configured to wirelessly couple to the neuromodulation device 100. The neuromodulation device 100 can include a lead 102 having a plurality of conductive elements 114 and an electronics package 108 having a first antenna 116 (also referred to herein as a power harvesting antenna 116) and an electronics component 118. In some variations, the electronics package 108 can further include a second antenna 120 (also referred to herein as a communications antenna 120). The neuromodulation device 100 is configured to be implanted at a treatment site comprising submental and sublingual regions of a patient's head, as detailed below with reference to FIGS. 3A-3F.

In use, the electronics package 108 or one or more elements thereof can be configured provide a stimulation energy to the conductive elements 114 that has a pulse width, amplitude, duration, frequency, duty cycle, and/or polarity such that the conductive elements 114 apply an electric field at the treatment site that modulates the hypoglossal nerve. The stimulation energy can be delivered according to a periodic waveform including, for example, a charge-balanced square wave comprising alternating anodic and cathodic pulses.

One or more pulses of the stimulation energy can have a pulse width between about 10 μs and about 1000 μs, between about 50 μs and about 950 μs, between about 100 μs and about 900 μs, between about 150 μs and about 800 μs, between about 200 μs and about 850 μs, between about 250 μs and about 800 μs, between about 300 μs and about 750 μs, between about 350 μs and about 700 μs, between about 400 μs and about 650 μs, between about 450 μs and about 600 μs, between about 500 μs and about 550 μs, about 50 μs, about 100 μs, about 150 μs, about 200 μs, about 250 μs, about 300 μs, about 350 μs, about 400 μs, about 450 μs, about 500 μs, about 550 μs, about 600 μs, about 650 μs, about 700 μs, about 750 μs, about 800 μs, about 850 μs, about 900 μs, about 950 μs, and/or about 1000 μs. In some embodiments, one or more pulses of the stimulation energy has a pulse width of between about 50 μs and about 450 μs.

One or more pulses of the stimulation energy can have an amplitude sufficient to cause an increase in phasic activity of a desired muscle. For example, one or more pulses of the stimulation energy can have a current-controlled amplitude between about 0.1 mA and about 5 mA. In some embodiments, the stimulation energy has an amplitude of about 0.3 mA, about 0.4 mA, about 0.5 mA, about 0.6 mA, about 0.7 mA, about 0.8 mA, about 0.9 mA, about 1 mA, about 1.5 mA, about 2 mA, about 2.5 mA, about 3 mA, about 3.5 mA, about 4 mA, about 4.5 mA, and/or about 5 mA. Additionally or alternatively, an amplitude of one or more pulses of the stimulation energy can be voltage-controlled. An amplitude of one or more pulses of the stimulation energy can be based at least in part on a size and/or configuration of the conductive elements 114, a location of the conductive elements 114 in the patient, etc.

A frequency of the pulses of the stimulation energy can be between about 10 Hz and about 50 Hz, between about 20 Hz and about 40 Hz, about 10 Hz, about 15 Hz, about 20 Hz, about 25 Hz, about 30 Hz, about 35 Hz, about 40 Hz, about 45 Hz, and/or about 50 Hz. In some embodiments, the frequency can be based on a desired effect of the stimulation energy on one or more muscles or nerves. For example, lower frequencies may induce a muscular twitch whereas higher frequencies may include complete contraction of a muscle.

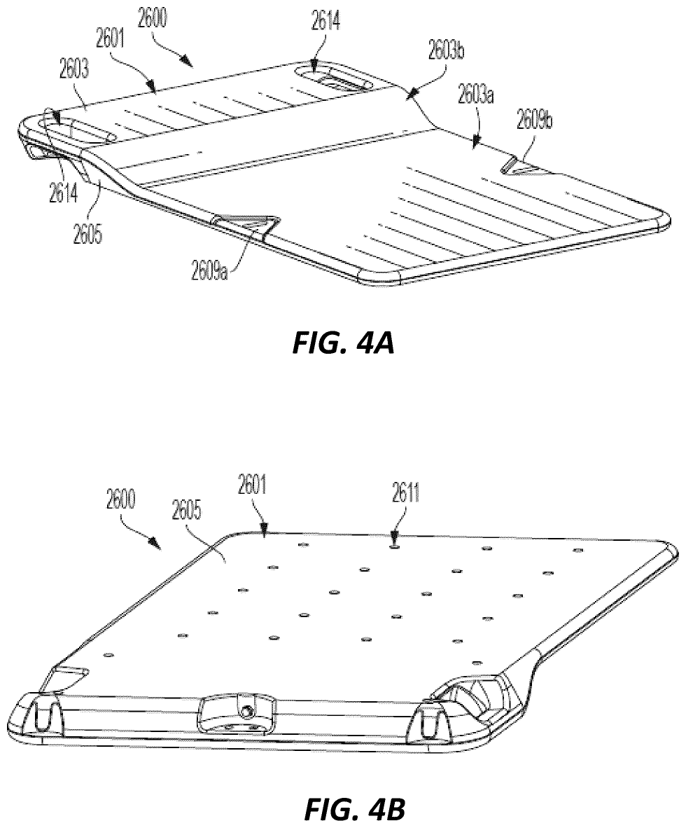

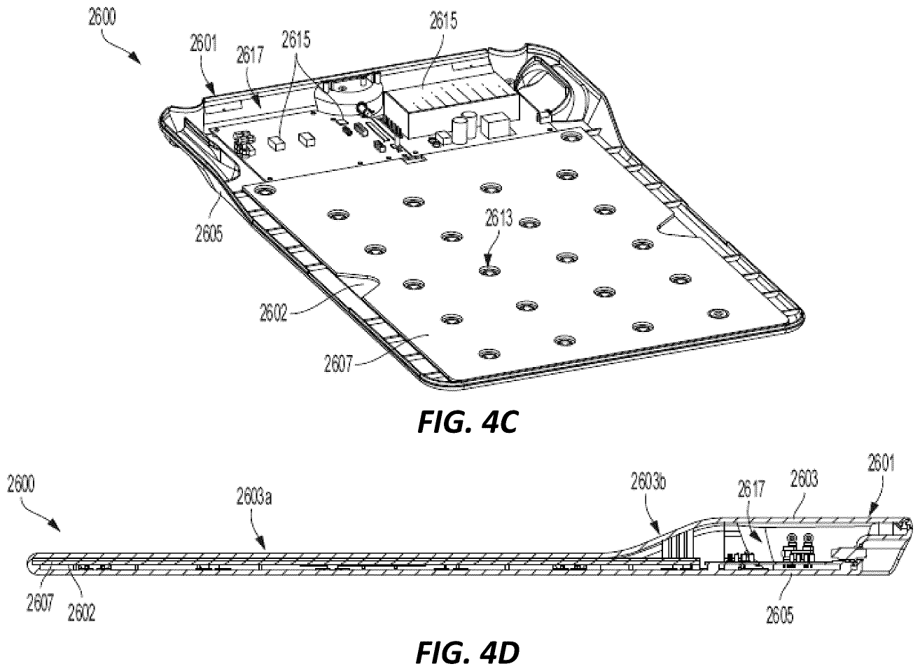

The external system 15 can comprise an external device 11 and a control unit 30 communicatively coupled to the external device 11. In some embodiments, the external device 11 is configured to be positioned proximate a patient's head while they sleep. The external device 11 can comprise a carrier 9 integrated with a first external antenna 12 and/or a second external antenna 20. Additional details regarding the external system 15 and the external device 11 are provided below with reference to FIGS. 4A-4D. While the control unit 30 is shown separate from the external device 11 in FIG. 2A, in some embodiments the control unit 30 can be integrated with and/or comprise a portion of the external device 11. The first external antenna 12 can be configured to power the neuromodulation device 100 through electromagnetic induction. For example, electrical current can be induced in the power harvesting antenna 116 when it is positioned above the first external antenna 12 of the external device 11, in an electromagnetic field produced by first external antenna 12. Furthermore, in embodiments in which the electronics package 108 of the neuromodulation system 100 includes a communications antenna 120, the second external antenna 20 can be configured to transmit data to and/or receive data from the communications antenna 120 in the neuromodulation device 100, via one or more wireless communication techniques (e.g., Bluetooth®, WiFi®, USB, etc.) to facilitate communication between the neuromodulation device 100 and the external system 15. This communication can, for example, include programming, e.g., uploading software/firmware revisions to the neuromodulation device 100, changing/adjusting stimulation settings and/or parameters, and/or adjusting parameters of control algorithms. Additionally or alternatively, in some embodiments, the power harvesting antenna 116 and the first external antenna 12 can be configured to transmit data to and/or receive data from one another via one or more wireless communication techniques.

The control unit 30 of the external system 15 can include a processor and/or a memory that stores instructions (e.g., in the form of software, code or program instructions executable by the processor or controller) for causing the external device to generate an electromagnetic field according to certain parameters provided by the instructions. The external system 15 or one or more portions thereof, such as the control unit 30, can include and/or be configured to be coupled to a power source such as a direct current (DC) power supply, an alternating current (AC) power supply, and/or a power supply switchable between DC and AC. The processor can be used to control various parameters of the energy output by the power source, such as intensity, amplitude, duration, frequency, duty cycle, and polarity. Instead of or in addition to a processor, the external system can include drive circuitry. In such embodiments, the external system 15 or one or more portions thereof (e.g., control unit 30), can include hardwired circuit elements to provide the desired waveform delivery rather than a software-based generator. The drive circuitry can include, for example, analog circuit elements (e.g., resistors, diodes, switches, etc.) that are configured to cause the power source to supply energy to the first external antenna 12 to produce an electromagnetic field according to the desired parameters. In some embodiments, the neuromodulation device 100 can be configured for communication with the external system via inductive coupling.

The system 10 can also include a user interface 40 in the form of a patient device 70 and/or a physician device 75. The user interface(s) 40 can be configured to transmit and/or receive data with the external system 15, the first external antenna 12, the second external antenna 20, the control unit 30, the neuromodulation device 100, and/or the remote computing device(s) 80 via wired and/or wireless communication techniques (e.g., Bluetooth, WiFi, USB, etc.). In the example configuration of FIG. 2A, both the patient device 70 and physician device 75 are smartphones. The type of device could, however, vary. One or both of the patient device 70 and physician device 75 can have an application or “app” installed thereon that is user specific, e.g., a patient app or a physician app, respectively. The patient app can allow the patient to execute certain commands necessary for controlling operation of neuromodulation device 100, such as, for example, start/stop therapy, increase/decrease stimulation power or intensity, and/or select a stimulation program. In addition to the controls afforded the patient, the physician app can allow the physician to modify stimulation settings, such as pulse settings (patterns, duration, waveforms, etc.), stimulation frequency, amplitude settings, and electrode configurations, closed-loop and open loop control settings and tuning parameters for the embedded software that controls therapy delivery during use.

The patient and/or physician devices 70, 75 can be configured to communicate with the other components of the system 10 via a network 50. The network 50 can be or include one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof. The patient and/or physician devices 70, 75 can be configured to communicate with one or more remote computing devices 80 via the network 50 to enable the transfer of data between the devices 70, 75 and the remote computing device(s) 80. Additionally, the external system 15 can be configured to communicate with the other components of the system 10 via the network 50. This can also enable the transfer of data between the external system 15 and remote computing device(s) 80.

The external system 15 can receive the programming, software/firmware, and settings/parameters through any of the communication paths described above, e.g., from the user interface(s) 40 directly (wired or wirelessly) and/or through the network 50. The communication paths can also be used to download data from the neuromodulation device 100, such as measured data regarding completed stimulation therapy sessions, to the external system 15. The external system 15 can transmit the downloaded data to the user interface 40, which can send/upload the data to the remote computing device(s) 80 via the network 50.

In addition to facilitating local control of the system 10, e.g., the external system 15 and the neuromodulation device 100, the various communication paths shown in FIG. 2A can also enable:
Distributing from the remoting computing device(s) 80 software/firmware updates for the patient device 70, physician device 75, external system 15, and/or neuromodulation device 100.
Downloading from the remote computing device(s) 80 therapy settings/parameters to be implemented by the patient device 70, physician device 75, external system 15, and/or neuromodulation device 100.
Facilitating therapy setting/parameter adjustments/algorithm adjustments by a remotely located physician.
Uploading data recorded during therapy sessions.
Maintaining coherency in the settings/parameters by distributing changes and adjustments throughout the system components.

The therapeutic approach implemented with the system 10 can involve implanting only the neuromodulation device 100 and leaving the external system 15 as an external component to be used only during the application of therapy. To facilitate this, the neuromodulation device 100 can be configured to be powered by the external system 15 through electromagnetic induction. In use, the first external antenna 12, operated by control unit 30, can be positioned external to the patient in the vicinity of the neuromodulation device 100 such that the first external antenna 12 is close to the power harvesting antenna 116 of the neuromodulation device 100. In some embodiments, the first external antenna 12 is carried by a flexible carrier 9 that is configured to be positioned on or sufficiently near the sleeping surface while the patient sleeps to maintain the position of the power harvesting antenna 116 within the target volume of the electromagnetic field generated by the first external antenna 12. Through this approach, the system 10 can deliver therapy to improve SDB (such as OSA), for example, by stimulating the HGN through a shorter, less invasive procedure. The elimination of an on-board, implanted power source in favor of an inductive power scheme can eliminate the need for batteries and the associated battery changes over the patient's life.

In some embodiments, the system 10 can include one or more sensors (not shown), which may be implanted and/or external. For example, the system 10 can include one or more sensors carried by (and implanted with) the neuromodulation device 100. Such sensors can be disposed at any location along the lead 102 and/or electronics package 108. In some embodiments, one, some, or all of the conductive elements 114 can be used for both sensing and stimulation. Use of a single structure or element as the sensor and the stimulating electrode reduces the invasive nature of the surgical procedure associated with implanting the system, while also reducing the number of foreign bodies introduced into a patient. In certain embodiments, at least one of the conductive elements 114 is dedicated to sensing only.

In addition to or instead of inclusion of one or more sensors on the neuromodulation device 100, the system 10 can include one or more sensors separate from the neuromodulation device 100. In some embodiments, one or more of such sensors are wired to the neuromodulation device 100 but implanted at a different location than the neuromodulation device 100. In some embodiments, the system 10 includes one or more sensors that are configured to be wirelessly coupled to the neuromodulation device 100 and/or an external computing device (e.g., control unit 30, user interface 40, etc.). Such sensors can be implanted at the same or different location as the neuromodulation device 100, or may be disposed on the patient's skin.

The one or more sensors can be configured to record and/or detect physiological data (e.g., data originating from the patient's body) over time including changes therein. The physiological data can be used to select certain stimulation parameters and/or adjust one or more stimulation parameters during therapy. Physiological data can include an electromyography (EMG) signal, temperature, movement, body position, electroencephalography (EEG), air flow, audio data, heart rate, pulse oximetry, eye motion, and/or combinations thereof. In some embodiments, the physiological data can be used to detect and/or anticipate other physiological parameters. For example, the one or more sensors can be configured to sense an EMG signal which can be used to detect and/or anticipate physiological events such as phasic contraction of anterior lingual musculature (such as phasic genioglossus muscle contraction) and measure physiological data such as underlying tonic activity of anterior lingual musculature (such as tonic activity of the genioglossus muscle). Phasic contraction of the genioglossus muscle can be indicative of inspiration, particularly the phasic activity that is layered within the underlying tonic tone of the genioglossus muscle. Changes in physiological data include changes in one or more parameters of a measured signal (e.g., frequency, amplitude, spike rate, etc.), start and end of phasic contraction of anterior lingual musculature (such as phasic genioglossus muscle contraction), changes in underlying tonic activity of anterior lingual musculature (such as changes in tonic activity of the genioglossus muscle), and combinations thereof. In particular, changes in phasic activity of the genioglossus muscle can indicate a respiration or inspiration change and can be used to trigger stimulation. Such physiological data and changes therein can be identified in signals recorded from sensors during different phases of respiration including inspiration. As such, the one or more sensors can include EMG sensors. The one or more sensors can also include, for example, wireless or tethered sensors that measure, body temperature, movement (e.g., an accelerometer), breath sounds (e.g., audio sensors), heart rate, pulse oximetry, eye motion, etc.

In operation, the physiological data provided by the one or more sensors enables closed-loop operation of the neuromodulation device 100. For example, the sensed EMG responses from the genioglossus muscle can enable closed-loop operation of the neuromodulation device 100 while eliminating the need for a chest lead to sense respiration. Operating in closed-loop, the neuromodulation device 100 can maintain stimulation synchronized with respiration, for example, while preserving the ability to detect and account for momentary obstruction. The neuromodulation device 100 can also detect and respond to snoring, for example.

The system 10 can be configured to provide open-loop control and/or closed-loop stimulation to configure parameters for stimulation. In other words, with respect to closed-loop stimulation, the system 10 can be configured to track the patient's respiration (such as each breath of the patient) and stimulation can be applied during or prior to onset of inspiration, for example. However, with respect to open-loop stimulation, stimulation can be applying without tracking specific physiological data, such as respiration or inspiration. However, even under such an “open loop” scenario, the system 10 can still adjust stimulation and record data, to act on such information. For example, one way the system 10 can act upon such information is that the system 10 can configure parameters for stimulation to apply stimulation in an open loop fashion but can monitor the patient's respiration to know when to revert to applying stimulation on a breath to breath, close-loop fashion such that the system 10 is always working in a closed-loop algorithm to assess data. Treatment parameters of the system may be automatically adjusted in response to the physiological data. The physiological data can be stored over time and examined to change the treatment parameters; for example, the treatment data can be examined in real time to make a real time change to the treatment parameters. In some embodiments, the treatment parameters can be learned from the physiological data stored over time and used to adjust the therapy in real time. This learning can be patient-specific and/or across multiple patients.

Operating in real-time, the neuromodulation device 100 can record data (e.g., via one or more sensors) related to the stimulation session including, for example, stimulation settings, EMG responses, respiration, sleep state including different stages of REM and non-REM sleep, etc. For example, changes in phasic and tonic EMG activity of the genioglossus muscle during inspiration can serve as a trigger for stimulation or changes in stimulation can be made based on changes in phasic and tonic EMG activity of the genioglossus muscle during inspiration or during different sleep states. This recorded data can be uploaded to the user interface 40 and to the remote computing device(s) 80. Also, the patient can be queried to use the interface 40 to log data regarding their perceived quality of sleep, which can also be uploaded to the remote computing device(s) 80. Offline, the remote computing device(s) 80 can execute a software application to evaluate the recorded data to determine whether settings and control parameters can be adjusted to further optimize the stimulation therapy. The software application can, for example, include artificial intelligence (AI) models that learn from recorded therapy sessions how certain adjustments affect the therapeutic outcome for the patient. In this manner, through AI learning, the model can provide patient-specific optimized therapy.

### III. Neuromodulation Devices

FIGS. 2B-2D illustrate various views of an example configuration of the neuromodulation device 100. While specific features of the neuromodulation device 100 are discussed with reference to FIGS. 2B-2D, other configurations of the neuromodulation device 100 are possible. Example configurations of neuromodulation devices 100 within the scope of the present technology include the neuromodulation devices found in U.S. Provisional Patent Application No. 63/377,969, filed Sep. 30, 2022, U.S. patent application Ser. No. 16/865,541, filed May 4, 2020, U.S. patent application Ser. No. 16/866,488, filed May 4, 2020, U.S. patent application Ser. No. 16/866,523, filed May 4, 2020, and U.S. patent application Ser. No. 16/865,668, filed May 4, 2020, each of which is incorporated in its entirety by this reference. As previously mentioned, the neuromodulation device 100 can be configured to be implanted at a treatment site within submental and sublingual regions of the patient's head and deliver electrical energy at the treatment site to stimulate the HGN and/or one or more tongue protruser muscles (e.g., the genioglossus, the geniohyoid, etc.). The neuromodulation device 100 can include an electronics package 108 and a lead 102 coupled to and extending away from the electronics package 108. The lead 102 can comprise a lead body 104 having a plurality of conductive elements 114 and an extension portion 106 extending between the lead body 104 and the electronics package 108. The extension portion 106 can have a proximal end portion 106a coupled to the electronics package 108 via a first connector 110 and a distal end portion 106b coupled to the lead body 104 via a second connector 112.

The electronics package 108 can be configured to supply electrical current to the conductive elements 114 (e.g., to stimulate) and/or receive electrical energy from the conductive elements 114 (e.g., to sense physiological data). The extension portion 106 of the lead 102 can mechanically and/or electrically couple the electronics package 108 to the lead body 104. The extension portion 106 can comprise a polymeric material such as, but not limited to, a thermoplastic elastomer, a thermoplastic polyurethane, a silicone, or other suitable materials. The extension portion 106 can be sufficiently flexible such that it can bend so as to position the lead body 104 on top of, but spaced apart from, the electronics package 108. As discussed in greater detail below with reference to FIGS. 3A-3F, the neuromodulation device 100 is configured to be implanted within both a submental region and a sublingual region such that the electronics package 108 and lead body 104 are vertically stacked with one or more muscle and/or other tissue layers positioned therebetween. The flexibility of the extension portion 106 enables such a configuration.

In some embodiments, the extension portion 106 comprises a sidewall defining a lumen extending through the extension portion 106. The conductive elements 114 can be electrically coupled to the first antenna 116 and/or the electronics component 118 via one or more electrical connections extending through the lumen of the extension portion 106. For example, the proximal end portions of the electrical connections can be routed through the first connector 110 to the electronics component 118 on the electronics package 108. The electrical connections may comprise, for example, one or more wires, cables, traces, vias, and others extending through the extension portion 106 and lead body 104. The electrical connections can comprise a conductive material such as silver, copper, etc., and each electrical connection can be insulated along all or a portion of its length. In some embodiments, the neuromodulation device 100 includes a separate electrical connection for each conductive element 114. For example, in those embodiments in which the neuromodulation device 100 comprises eight conductive elements 114 (and other embodiments), the neuromodulation device 100 can comprise eight electrical connections, each extending through the lumen of the extension portion 106 from a proximal end at the electronics component 118 to a distal end at one of the conductive elements 114.

In some embodiments, the electronics component 118 comprise an application-specific integrated circuit (ASIC), a discrete electronic component, and/or an electrical connector. In these and other embodiments, the electronics component 118 can comprise, for example, processing and memory components (e.g., microcomputers, microprocessors, computers-on-a-chip, etc.), charge storage and/or delivery components (e.g., batteries, capacitors, electrical conductors) for receiving, accumulating, and/or delivering electrical energy, switching components (e.g., solid state, pulse-width modulation, etc.) for selection and/or control of the conductive elements 114. In some embodiments, the electronics component 118 comprise a data communications unit for communicating with an external device (such as external system 15) via a communication standard such as, but not limited to, near-field communication (NFC), infrared wireless, Bluetooth, ZigBee, Wi-Fi, inductive coupling, capacitive coupling, or any other suitable wireless communication standard. In some examples, the electronics component 118 includes one or more processors having one or more computing components configured to control energy delivery via the conductive elements 114 and/or process energy and/or data received by the conductive elements 114 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the conductive elements 114 to obtain data characterizing activity of a patient's muscles. In another example, the functions may involve processing data to determine one or more parameters of the data (e.g., a change in muscle activity, etc.). According to various embodiments, the electronics component 118 can comprise a wireless charging unit for providing power to other electronics component 118 of the neuromodulation device 100 and/or recharging a battery of the neuromodulation device 100 (if included).

The electronics package 108 can also be configured to wirelessly receive energy from a power source to power the neuromodulation device 100. In some embodiments, the electronics package 108 comprises a power harvesting antenna 116 and a communications antenna 120 configured to wirelessly communicate with the external system 15, along with an electronics component 118. As shown in FIG. 2B, in some embodiments the electronics component 118 can be disposed in an opening at a central portion of the power harvesting antenna 116 and/or in an opening at a central portion of the communications antenna 120. In other embodiments, the electronics component 118 may have another configuration and/or arrangement relative to the power harvesting antenna 116 and the communications antenna 120.

With continued reference to FIGS. 2B-2D, the lead body 104 can comprise a substrate carrying one or more conductive elements 114 configured to deliver and/or receive electrical energy. In some embodiments, the lead body 104 (or one or more portions thereof) comprises flexible tubing with a sidewall defining a lumen. The lead body 104 can comprise a polymeric material such as, but not limited to, a thermoplastic elastomer, a thermoplastic polyurethane, a silicone, or other suitable materials. The lead body 104 can comprise the same material as the extension portion 106 or a different material. The lead body 104 can comprise the same material as the extension portion 106 but with a different durometer. In some embodiments, the lead body 104 has a lower durometer than the extension portion 106, which can enhance patient comfort.

As shown in FIGS. 2B-2D, the lead body 104 has a branched shape comprising a first arm 122 and a second arm 124. To facilitate this configuration, for example, the second connector 112 can be bifurcated and/or branching. The first arm 122 and the second arm 124 can each extend distally and laterally from the second connector 112 and/or the distal end portion 106b of the extension portion 106. The first arm 122 can comprise a proximal portion 122a, a distal portion 122b, and an intermediate portion 122c extending between the proximal portion 112a and the distal portion 122b. Similarly, the second arm 124 can comprise a proximal portion 124a, a distal portion 124b, and an intermediate portion 124c extending between the proximal portion 124a and the distal portion 124b. In some embodiments, the first arm 122 can comprise a cantilevered, free distal end 123 and/or the second arm 124 can comprise a cantilevered, free distal end 125. The first arm 122 and/or the second arm 124 can include one or more fixation elements 130, for example the fixation elements 130 shown at the distal end portions 122b, 124b of the first and second arms 122, 124 in FIGS. 2B-2D. The fixation elements 130 can be configured to securely, and optionally releasably, engage patient tissue to prevent or limit movement of the lead body 104 relative to the tissue.

While being flexible, the lead 102 and/or one or more portions thereof (e.g., the lead body 104, the extension portion 106, etc.) can also be configured to maintain a desired shape. This feature can, for example, be facilitated by electrical conductors that electrically connect the conductive elements 114 carried by the lead body 104 to the electronics package 108, by an additional internal shape-maintaining (e.g., a metal, a shape memory alloy, etc.) support structure (not shown), by shape setting the substrate comprising the lead 102, etc. In any case, one or more portions of the lead 102 can have a physical property (e.g., ductility, elasticity, etc.) that enable the lead 102 to be manipulated into a desired shape or maintain a preset shape. Additionally or alternatively, the lead 102 and/or one or more portions thereof (e.g., the lead body 104, the extension portion 106, etc.) can be sufficiently flexible to at least partially conform to a patient's anatomy once implanted and/or to enhance patient comfort.

The conductive elements 114 can be carried by the sidewall of the lead body 104. For example, the conductive elements 114 can be positioned on an outer surface of the sidewall and/or within a recessed portion of the sidewall. In some embodiments, one or more of the conductive elements 114 is positioned on an outer surface of the sidewall and extends at least partially around a circumference of the sidewall. The lumen of the lead body 104 can carry one or more electrical conductors that extend through the lumen of the lead body 104 and the lumen of the extension portion 106 from the conductive elements 114 to the electronics package 108. The sidewall can define one or more apertures through which an electrical connector can extend.

Each of the conductive elements 114 may comprise an electrode, an exposed portion of a conductive material, a printed conductive material, and other suitable forms. In some embodiments, one or more of the conductive elements 114 comprises a ring electrode. The conductive elements 114 can be crimped, welded, adhered to, or positioned over an outer surface and/or recessed portion of the lead body 104. Additionally or alternatively, each of the conductive elements 114 can be welded, soldered, crimped, or otherwise electrically coupled to a corresponding electrical connector. In some embodiments, one or more of the conductive elements 114 comprises a flexible conductive material disposed on the lead body 104 via printing, thin film deposition, or other suitable techniques. Each one of the conductive elements 114 can comprise any suitable conductive material including, but not limited to, platinum, iridium, silver, gold, nickel, titanium, copper, combinations thereof, and/or others. For example, one or more of the conductive elements 114 can be a ring electrode comprising a platinum iridium alloy. In some embodiments, one or more of the conductive elements 114 comprises a coating configured to improve biocompatibility, conductivity, corrosion resistance, surface roughness, durability, or other parameter(s) of the conductive element 114. As but one example, one or more of the conductive elements 114 can comprise a coating of titanium and nitride.

In some embodiments, one or more conductive elements 114 has a length of about 1 mm. Additionally or alternatively, one or more conductive elements 114 can have a length of about 0.25 mm, about 0.5 mm, about 0.75 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. In any case, adjacent conductive elements 114 carried by one of the first or second arms 122, 124 can be spaced apart along a length of the arm by about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. The conductive elements 114 can have the same length or different lengths.

While the neuromodulation device 100 shown in FIGS. 2B-2D includes eight conductive elements 114 (four conductive elements 114 carried by the first arm 122 and four conductive elements 114 carried by the second arm 124), other numbers and configurations of conductive elements 114 are within the scope of the present technology. For example, the first arm 122 can carry the same number of conductive elements 114 as the second arm 124, or the first arm 122 can carry a different number of conductive elements 114 as the second arm 124. The first arm 122 and/or the second arm 124 can carry one conductive element 114, two conductive elements 114, three conductive elements 114, four conductive elements 114, five conductive elements 114, six conductive elements 114, seven conductive elements 114, eight conductive elements 114, nine conductive elements 114, ten conductive elements 114, or more than ten conductive elements 114. In some embodiments, one of the first arm 122 or the second arm 124 does not carry any conductive elements 114.

The conductive elements 114 can be configured for stimulation and/or sensing. Stimulating conductive elements 114 can be configured to deliver energy to an anatomical structure, such as, for example, a nerve or muscle. In some embodiments, the conductive elements 114 are configured to deliver energy to a hypoglossal nerve of a patient to increase the activity of the patient's tongue protrusor muscles. Sensing conductive elements 114 can be used obtain data characterizing a physiological activity of a patient (e.g., muscle activity, temperature, etc.). In some embodiments, the sensing conductive elements 114 are configured to detect electrical energy produced by a muscle of a patient to obtain EMG data characterizing an activity of the muscle. In some embodiments, the sensing conductive elements are configured to measure impedance across the conductive elements. As but one example, in some embodiments the conductive elements 114 are configured to deliver energy to a hypoglossal nerve of a patient to increase activity of the genioglossus and/or geniohyoid muscles, and obtain EMG data characterizing activity of the genioglossus muscle and/or the geniohyoid muscle of the patient. Still, the conductive elements 114 can be configured to deliver energy to and/or measure physiological electrical signals from other patient tissues.

The function that each of the conductive elements 114 is configured to perform (e.g., delivering energy to patient tissue, receiving energy from patient tissue, etc.) can be controlled by a processor of the electronics component 118 of the electronics package 108. In some embodiments, one or more of the conductive elements 114 is configured for only one of delivering energy to patient tissue or receiving energy from patient tissue. In various embodiments, one or more of the conductive elements 114 is configured for both delivering energy to patient tissue and receiving energy from patient tissue. In some embodiments, the functionality of a conductive element 114 can be based, at least in part, on an intended positioning of the neuromodulation device 100 within a patient and/or the position of the conductive element 114 on the lead body 104. One, some, or all of the conductive elements 114 can be positioned relative to patient tissue, such as nerves and/or muscles, so that it may be desirable for the conductive element(s) 114 to be able to both deliver energy to the patient tissue and receive energy from the patient tissue. Additionally or alternatively, some conductive elements 114 can have an intended position relative to specific patient tissues so that only delivery of stimulation energy is desired while other conductive elements 114 can have an intended position relative to specific patient tissues so that only receipt of sensing energy is desired. Advantageously, the configurations of the conductive elements 114 can be configured in software settings (which can be facilitated by electronics component 118 of the electronics package 108) so that the configurations of the conductive elements 114 are easily modifiable.

Whether configured for stimulating and/or sensing, each of the conductive elements 114 can be configured and used independently of the other conductive elements 114. Because of this, all or some of conductive elements 114, whichever is determined to be most effective for a particular implementation, can be utilized during the application of stimulation therapy. For example, one conductive element 114 of the first arm 122 can be used as a cathode while one conductive element 114 of the second arm 124 is used as an anode (or vice versa), two or more conductive elements 114 of the first arm 122 can be used (one as the cathode and one as the anode) without use of any conductive elements 114 of the second arm 124 (or vice versa), multiple pairs of conductive elements 114 of the first and second arms 122, 124 can be used, or any other suitable combination. The conductive element(s) 114 used for sensing and/or stimulation can be selected based on desired data to be collected and/or desired modulation of neural or muscle activity. For example, specific pairs of the conductive elements 114 can be used for creating an electric field tailored to stimulation of certain regions of the muscle and/or HGN that causes favorable changes in tongue position and/or pharyngeal dilation. Additionally or alternatively, conductive element(s) 114 that are positioned in contact with muscle tissue when the neuromodulation device 100 is implanted may be more favorable to use for EMG sensing than conductive element(s) 114 that are not positioned in contact with muscle tissue.

The lead body 104 can have a shape configured to facilitate delivery of electrical energy to a specific treatment location within a patient and/or detection of electrical energy from a sensing location within the patient. The conductive elements 114 carried by the first arm 122 can be configured to deliver electrical stimulation energy to one hypoglossal nerve (e.g., the right or the left hypoglossal nerve) of a patient and the conductive elements 114 carried by the second arm 124 can be configured to deliver electrical stimulation energy to the other hypoglossal nerve (e.g., the other of the right or the left hypoglossal nerve) of the patient.

Without being bound by theory, it is believed that increased activity of the tongue protrusor muscles during sleep reduces upper airway resistance and improves respiration. Thus, devices of the present technology are configured to deliver stimulation energy to motor nerves that control the tongue protrusors. In some embodiments, the neuromodulation device 100 is configured to deliver stimulation energy to the hypoglossal nerve to cause protrusion of the tongue. Additionally or alternatively, the neuromodulation device 100 can be configured to receive sensing energy produced by activity of one or more muscles of a patient (such as the genioglossus muscle), which can be used for closed-loop delivery of stimulation energy, evaluation of patient respiration, etc.

The device can be configured to be implanted at an anatomical region of a patient that is bound anteriorly and laterally by the patient's mandible, superiorly by the superior surface of the tongue, and inferiorly by the patient's platysma. Such an anatomical region can include, for example, a submental region and a sublingual region. The sublingual region can be bound superiorly by the oral floor mucosa and inferiorly by the mylohyoid and includes the plane between the genioglossus muscle and the geniohyoid muscle. The submental region can be bound superiorly by the mylohyoid and inferiorly by the platysma muscle. FIGS. 3A-3F depict various views of the neuromodulation device 100 implanted within a patient. As shown in FIGS. 3A-3F, the neuromodulation device 100 is configured to be positioned such that the electronics package 108 is disposed on or near the inferior surface of the mylohyoid in a submental region while the lead body 104 is positioned between the geniohyoid and genioglossus in a sublingual region with the arms 122, 124 disposed along the left and right hypoglossal nerves. The arms 122, 124 can be positioned such that the conductive elements 114 are disposed near the distal arborization of the hypoglossal nerves that innervate the genioglossus. In particular, the conductive elements 114 can be positioned proximate the portions of the distal arborization that innervate the horizontal fibers of the genioglossus while limiting and/or avoiding stimulation of the portions of the distal arborization of the hypoglossal nerve that activate retrusor muscles. When implanted, the extension portion 106 of the lead 102 can extend in an anterior direction away from the electronics package 108 (towards the mandible), then bend superiorly and extend through the geniohyoid muscle until bending back posteriorly and extending within a tissue plane between the geniohyoid and genioglossus muscles. In some embodiments, the extension portion 106 straddles the right and left geniohyoid muscles.

The electronics package 108 can be sufficiently flexible so that, once implanted, the electronics package 108 at least partially conforms to the curvature of the mylohyoid. Additionally or alternatively, the electronics package 108 can have a shape reflecting the curvature of the mylohyoid. In some embodiments, the electronics package 108 can comprise fixation elements (similar to fixation elements 130 or otherwise) that are configured to engage the mylohyoid (or other surrounding tissue) and prevent or limit motion of the electronics package 108 once implanted.

The lead body 104 can be configured to be positioned between the genioglossus and geniohyoid muscles of a patient so that the conductive elements 114 are positioned proximate the hypoglossal nerve. Although not shown in FIGS. 3A-3F, the hypoglossal nerve is located between the genioglossus and fascia and/or fat located between the genioglossus and the geniohyoid. In some embodiments, the lead body 104 is configured to be positioned at or just inferior to the fat between the hypoglossal nerve and the geniohyoid and thus is not positioned in direct contact with the hypoglossal nerve. In any case, once the neuromodulation device 100 is implanted, the lead body 104 can extend posteriorly away from the distal end portion 106b of the extension portion 106. The lead body 104 can then branch laterally such that the first arm 122 of the lead body 104 is positioned proximate one of the patient's hypoglossal nerves and the second arm 124 is positioned proximate the contralateral hypoglossal nerve. The fixation elements 130 can engage patient tissue (e.g., the fat underlying the hypoglossal nerves, etc.) to prevent or limit motion of the first and second arms 122, 124 relative to the patient tissue.

As best shown in FIG. 3C, the arms 122, 124 of the lead body 104 can bend out of the plane of the extension portion 106, in addition to extending laterally away from the extension portion 106, such that the arms 122, 124 outline a somewhat concave shape. Advantageously, this concave shape can accommodate the convex inferior surface of the genioglossus and still keep the arms 122, 124 positioned near the distal arborization of the hypoglossal nerve.

In some embodiments, conductive elements 114 are selected for use that selectively activate the protrusor muscles of a patient. In these and other embodiments, the specific positioning of the first and second arms 122, 124 relative to specific branches of the hypoglossal nerves need not be identified prior to stimulation of desired portions of the nerve and/or muscle. For example, in embodiments in which the lead body 104 includes more than two conductive elements 114, the combination of conductive elements 114 that is used for treating a patient can be selected based on physiological responses to test stimulations. For example, stimulation energy can be delivered to the hypoglossal nerve(s) via multiple combinations of conductive elements 114 and a physiological response (e.g., EMG data, tongue position, pharyngeal opening size, etc.) and/or a functional outcome (e.g., Fatigue Severity Scale, Epworth Sleepiness Scale, etc.) can be evaluated for each combination. Based on the evaluation(s), the conductive elements 114 that are selected to deliver stimulation energy can be conductive elements 114 that are associated with favorable responses/outcomes.

### IV. External Devices for Neuromodulation Systems

FIGS. 4A-4D illustrate an example of an external device 2600 configured for use in a clinical environment. Features of the external device 2600 can be generally similar to the features of the external device 11 of FIG. 2A. Any of the features of the external device 2600 of FIGS. 4A-4D can be combined with each other and/or with the features of the external device 11 of FIG. 2A and any of the features of the external device 11 of FIG. 2A can be combined with any of the features of the external device 2600 of FIGS. 4A-4D. Moreover, although the external device 2600 is described with reference to use in a clinical environment, the external device 2600 can be used in any environment or use case.

The external device 2600 can comprise a carrier 2601 enclosing a substrate carrying an external antenna, which can be similar to first external antenna 12 described above with reference to FIG. 2A for generating an electromagnetic field. FIGS. 4A and 4B are perspective views of the carrier 2601 of the external device 2600, and FIGS. 4C and 4D are cutaway views of the external device 2600.

As shown in FIGS. 4A and 4B, in some embodiments the carrier 2601 comprises an upper portion 2603 and a lower portion 2605. The upper and lower portions 2603, 2605 can be integral with one another, permanently secured to one another, and/or releasably secured to one another. The lower portion 2605 can be configured to be positioned on a surface beneath a patient (e.g., an operating table, an examination table, a sleeping surface, etc.). At least one region of the upper portion 2603 can be configured to be positioned between the lower portion 2605 of the carrier 2601 and the patient. As shown in FIG. 4B, the lower portion 2605 can be substantially flat. In some embodiments, the lower portion 2605 of the carrier 2601 defines one or more apertures 2611 (see FIG. 4B) each configured to receive a fastener therein for securing the carrier 2601 to the substrate 2602.

In some embodiments, for example as shown in FIG. 4A, the upper portion 2603 can comprise a substantially flat region 2603a and a ramped region 2603b. As shown in FIG. 4C, a substrate 2602 carrying the external antenna (not shown) can be positioned at the substantially flat region 2603a. The flat region 2603a and thereby the substrate 2602 and antenna 2604 can be configured to be positioned between the lower portion 2605 of the carrier 2601 and the patient. The ramped region 2603b and the lower portion 2605 of the carrier 2601 define an interior volume 2617 that is larger than an interior volume defined by the substantially flat region 2603a of the upper portion 2603 and the lower portion 2605 and/or has a larger cross-sectional area than a cross-sectional area defined by the substantially flat region 2603a. One or more electronic components 2615 (e.g., a control unit, a power source, etc.) can be positioned within the interior volume 2617 (see FIGS. 4C and 4D). The ramped region 2603b can be configured to be positioned proximate a patient but not underneath the patient during use. For example, the substantially flat region 2603a can be configured to be positioned between a patient's head and a surface beneath the patient while the ramped region 2603b is positioned laterally or superiorly of the patient's head.

Although FIGS. 4A-4D illustrate the device 2600 comprising a single carrier 2601 for carrying the substrate 2602 and electronic components 2615, in some embodiments the device 2600 comprises multiple, distinct carriers. For example, the device 2600 can comprise a first carrier 2601 carrying the substrate 2602 and a second, separate carrier 2601 carrying the electronic components 2615. The antenna 2604 can be electrically coupled to the electronic components 2615 via a connector extending between the first and second carriers. Separating the antenna 2604 from the electronic components 2615 may allow the electronic components 2615 to be positioned further away from the patient's head and/or body, thus preventing or limiting heat transfer from the electronics components 2615 to the patient. Moreover, each of the distinct carriers 2601 can comprise a distinct material based on the specific requirements of the individual carrier.

In some embodiments, the device 2600 can include cushioning. For example, cushioning can be carried by the carrier 2601 of the device so that, when the device 2600 is positioned proximate the patient during use, the cushioning is positioned between the carrier 2601 and the patient's head. While the patient may be asleep during use of the device 2600, the patient may still experience discomfort once awake if the patient has been laying on a hard surface for an extended time. Accordingly, the cushioning can have a low hardness parameter to improve patient comfort. The cushioning can be carried by the substantially flat region 2603a and/or the ramped region 2306b. The cushioning can comprise any sufficiently soft material, such as one or more foams. Additionally or alternatively, the cushioning can be configured to dissipate heat from the patient's head.

The external device 2600 can include one or more manipulation portions 2614 configured to facilitate manipulation of the external device 2600 by a user. For example, as shown in FIG. 4A, the manipulation portions 2614 can comprise apertures in the carrier 2601 that form handles that can be grasped by a user. In a clinical environment in which the external device 2600 is used on an anesthetized patient, use of the manipulation portions 2614 can allow a clinician to more easily grasp the external device 2600 and reposition the external device 2600 relative to the patient.

The external device 2600 can be configured for use during a procedure in which an implantable device is implanted in a patient's head, which may occur in an operating room while the patient lies on an operating table. Operating tables often comprise a substantial amount of metal and/or other materials such as carbon fiber that can modify an impedance of the external antenna. To prevent or limit the operating table from changing an impedance of the external antenna, the external device 2600 can include a shielding material. The shielding material can be positioned between the external antenna and lower portion 2605 of the carrier 2601 and/or can be configured to be positioned between the lower portion 2605 of the carrier 2601 and the operating table.

In some embodiments, the external antenna is driven with more power when used in an operating room relative to an at-home setting. Driving the external antenna with additional power can compensate for additional detuning and/or preloading of the external antenna that may occur in the operating room. When higher power is used to drive the external antenna, the control unit and/or the external antenna may produce more heat. To address this concern, the external device 2600 can include an insulation 2607 that modifies and/or controls heat dissipation from the external antenna. As shown in FIGS. 4C and 4D, the insulation 2607 can be positioned between the substrate 2602 carrying the external antenna, and the upper portion 2603 of the carrier 2601 to prevent or limit heat transfer from the external antenna to the patient. Insulation 2607 can be provided at any other suitable location within and/or on the carrier 2601 in addition to or in place of the insulation 2607 shown in FIGS. 4C and 4D. In some embodiments, the insulation 2607 comprises a substantially flat sheet. The insulation 2607 can define one or more apertures 2613 (see FIG. 4C) each configured for receiving a fastener therethrough for securing the insulation 2607 to the carrier 2601 via the fasteners. In various embodiments, the insulation 2607 does not include apertures 2613. The insulation 2607 can comprise synthetic fibers with high heat resistance such as aramid fibers, for example, and/or another suitable felt or insulative material. The insulation 2607 can be resistant to moisture absorption, in some embodiments.

The substrate 2602 is positioned between the upper portion 2603 and the lower portion 2605 of the carrier 2601. In some embodiments, the substrate 2602 is positioned between the lower portion 2605 and the insulation 2607. The substrate 2602 can comprise a printed circuit board (PCB) substrate. For example, the substrate 2602 can comprise FR4, CEM1, CEM3, FR2, PET, elastomers, and/or another suitable PCB substrate. The substrate 2602 can comprise a dielectric material with good heat resistance. In some embodiments, a substrate configured for use in a clinical setting is more rigid than a substrate configured for use in an at-home setting. The substrate 2602 can define one or more apertures 2616 extending therethrough. Similar to the apertures 2613 of the insulation 2607, the apertures 2616 of the substrate 2602 can each be configured to receive a fastener therein for securing the substrate 2602 to the carrier 2601. The apertures 2616 can be configured to receive columns, for example, of the carrier 2601 to secure the carrier 2601 to the substrate 2602. In some embodiments, the apertures 2613 can be configured to be bonded, adhered, welded, or otherwise fixed to such columns. According to various embodiments, the substrate 2602 can define the same number of apertures 2616 as the number of apertures 2613 defined by the insulation 2607 and/or the apertures 2616 of the substrate 2602 can be configured to align with the apertures 2613 of the insulation 2607. As a result, one fastener can extend through an aperture 2616 of the substrate 2602 and an aperture 2613 of the insulation 2607.

To facilitate powering the implantable power harvesting antenna with an electromagnetic field having a smaller active volume, the carrier 2601 of the external device 2600 can comprise one or more markings configured to facilitate positioning of the patient on/over the external device 2600. For example, FIG. 4A depicts the carrier 2601 comprising a first marking 2609a and a second marking 2609b (collectively “markings 2609”), which indicate a region of the external device 2600 at which an anatomical landmark of a patient (e.g., a chin, a nose, an car, etc.) should be positioned. The markings 2609 can comprise a recessed portion of the carrier 2601, a protruding portion of the carrier 2601, a discrete element secured to the carrier 2601, a printed material disposed on the carrier 2601, etc.

Additional details of suitable example external devices 11 (e.g., mats) are described in U.S. Provisional Application No. 63/483,961 filed Feb. 8, 2023, which is incorporated herein in its entirety by this reference.

### V. Selected Examples of Electronics Packages for Neuromodulation Devices

As previously described with reference to FIG. 2A, the neuromodulation systems 10 of the present technology can include an implantable neuromodulation device 100 with an electronics package 108 including a power harvesting antenna 116 and a communications antenna 120.

Generally, the power harvesting antenna 116 may be configured to wirelessly couple to the external antenna 12 of an external device 11 (e.g., similar to external device 2600 described above with reference to FIGS. 4A-4D). The external antenna 12 may be communicatively coupled to a control unit 30 of the external system 15. The control unit 30 delivers electrical current to the first external antenna 12, and current flows through the first external antenna 12 such that the first external antenna 12 generates an electromagnetic field. When the power harvesting antenna 116 of the neuromodulation device 100 is positioned within such an electromagnetic field, an electromotive force is induced in the power harvesting antenna 116, thereby inducing an electrical current in the power harvesting antenna 116, which can be used to provide power for operation of the neuromodulation device 100.

As described herein, the neuromodulation device 100 of the present technology can be configured to deliver stimulation energy to a treatment site within a patient while the patient is sleeping to stimulate the HGN and/or the genioglossus muscle to improve the patient's respiration during sleep. Accordingly, the external system 15 can be configured to provide power to the implantable neuromodulation device 100 while a patient is sleeping to operate the implantable neuromodulation device 100. The external device 11 can be configured to be positioned between a patient's body and a sleeping surface that the patient lays upon while sleeping. For example, the external device 11 can be configured to be positioned between the patient's head, neck, upper back, and/or another anatomical region and a surface of the patient's mattress. The first external antenna 12 in the external device 11 can be configured to generate an electromagnetic field having a specific amplitude and distribution such that, when the patient is positioned proximate the external device 11, the electromagnetic field provides operational power to the neuromodulation device 100 located at a treatment site comprising submental and sublingual regions of a patient's head. According to various embodiments, operational power comprises between about 5 mW to about 50 mW, but may vary based on power requirements of the neuromodulation device 100, a size of the power harvesting antenna 116, a design of the power harvesting antenna 116, etc.

Power is delivered to the neuromodulation device 100 from the external system 15 by positioning the power harvesting antenna 116 within the electromagnetic field generated by the first external antenna 12, which induces an electromotive force in the power harvesting antenna 116. Specifically, the change in the magnetic flux (e.g., the amount of magnetic field perpendicularly penetrating the power harvesting antenna 116) induces an electromotive force in the power harvesting antenna 116. Accordingly, it is the component of the magnetic field parallel to the axial dimension of the power harvesting antenna 116 that induces an electromotive force in the power harvesting antenna 116. Because the power harvesting antenna 116 of the neuromodulation device 100 is configured to be positioned within a submental region just inferior of a patient's mylohyoid, the plane of power harvesting antenna 116 can be generally aligned with a transverse plane of the patient. Therefore, the first external antenna 12 can be configured to generate a magnetic field having a component substantially perpendicular to the transverse plane of the patient for delivering power to the neuromodulation device 100.

Furthermore, generally, the communications antenna 120 functions to transmit data to and/or receive data from the second external antenna 20 in the external system 15 (e.g., external device 11, such as a mat) via one or more wireless communication techniques (e.g., Bluetooth®, WiFi®, USB, etc.). For example, in some embodiments, the communications antenna 120 can include a network interface that is compatible with IEEE 802.15, such as Bluetooth®. This wireless communication facilitates communication between the neuromodulation device 100 and the external system 15, which can, for example, include programming (e.g., uploading software/firmware revisions to the neuromodulation device 100), changing/adjusting stimulation settings and/or parameters, and/or adjusting parameters of control algorithms.

In some embodiments in accordance with the present technology, the power harvesting antenna 112 and the communications antenna 120 are located on the same circuit board or integrated circuit board assembly (referred to collectively herein as a circuit board or a flexible circuit, though the circuit board may in some embodiments be rigid or semi-rigid). In many embodiments, it may be advantageous for the power harvesting antenna 112 and the communications antenna 120 to be included on the same circuit board. For example, a device including such an antenna arrangement may be configured to have a smaller form factor and/or fewer separate components, which for an implantable device may advantageously allow for less invasive implantation techniques and/or less interference with surrounding anatomy, thereby reducing adverse patient experiences and/or outcomes. As an illustrative example, when in an electronics package 108 of a neuromodulation device 100, this antenna arrangement allows for a more compact neuromodulation device 100 that is more suited for its intended implantation location as described above with respect to FIGS. 3A-3F. Specifically, this antenna arrangement on a flexible circuit in an electronics package 108 enables the electronics package 108 to have a suitable form factor and conformability to be placed in the limited anatomical space on or near the inferior surface of the mylohyoid in a submental region. This antenna arrangement contrasts with existing conventional neuromodulation devices, which typically include a conductive canister (“can”) that houses a power harvesting antenna of the device, and separately includes a communications antenna in a distinct epoxy header that extends from the can.

For at least the reasons described above, it can be more advantageous for a device such as neuromodulation device 100 to have an antenna arrangement with the power harvesting antenna 112 and the communications antenna 120 on the same circuit board. However, for such a device to function effectively, the electronics package 108 must overcome a number of challenges that are not faced by, and thus are not addressed by, existing conventional neuromodulation devices. For example, the power harvesting antenna 112 may create eddy currents that are picked by the nearby communications antenna 120 and thus interfere with the communications signal (e.g., Bluetooth signal) to and/or from the communications antenna 120. As another example, conductive features such as ground plates in the circuit board may couple eddy currents from the power harvesting antenna 112, thereby further attenuating the communications signal.

Furthermore, due to environmental factors surrounding the communications antenna 120 once the electronics package 108 is implanted in a patient, the signal from the communications antenna 120 can be significantly attenuated and have reduced signal quality. For example, when the neuromodulation device 100 is in operation, a signal generated by the communications antenna 120 may pass through substrate material of the electronics package 108, any material surrounding the substrate (e.g., encapsulation material), various tissues of the patient in which the electronics package 108 is implanted, and finally through air external to the patient to reach an external antenna such as in external device 11. Each of these materials has different electrical properties (e.g., conductivity, relative permittivity, etc.) that affect the ability of the communications signal to propagate through the material. As a result, the communications signal from the communications antenna 120 may be attenuated to different degrees as it travels through each material and across each interface between distinct materials, thereby reducing signal quality and/or efficiency of communications.

Examples of electronics package 108 described herein are configured to address various challenges associated with an antenna arrangement that includes the power harvesting antenna 112 and the communications antenna 120 on the same circuit board, as well as various challenges relating to attenuation of the signal from the communications antenna 120.

### 1. Substrate Structure

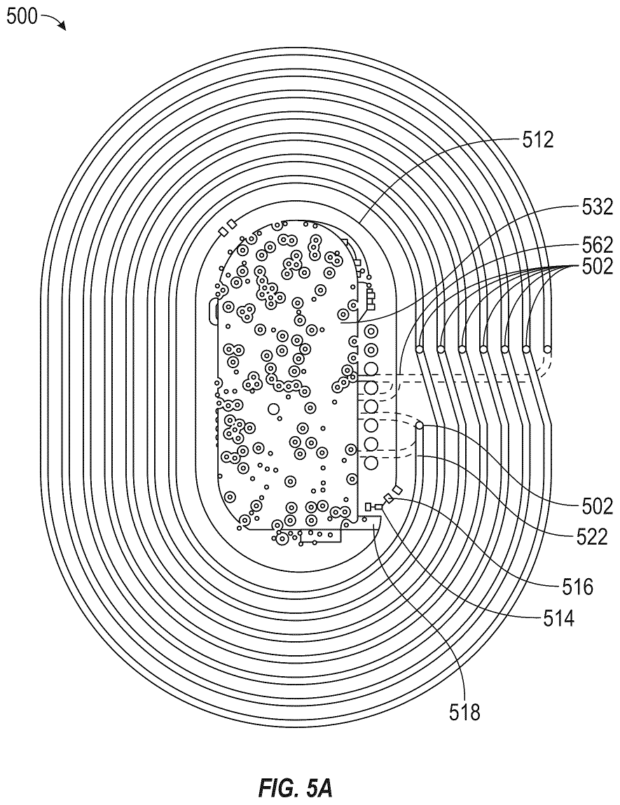

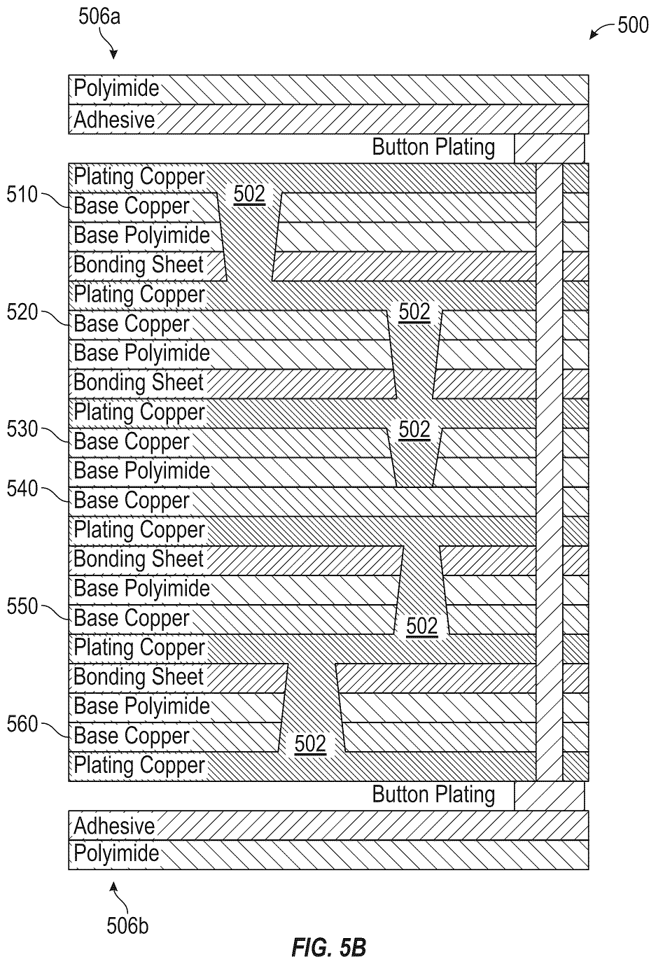

As shown in FIG. 5A, in some embodiments, an electronics package can include a flexible circuit 500 including a substrate carrying a power harvesting antenna 522 configured to operate at a first frequency and a communications antenna 512 configured to operate at a second frequency different from the first frequency. The electronics package can be similar to the electronics package 108 as described elsewhere herein. Furthermore, the power harvesting antenna 522 can be similar to the power harvesting antenna 116 as described elsewhere herein, and the communications antenna 512 can be similar to the communications antenna 120 as described elsewhere herein. For example, similar to the power harvesting antenna 116, the power harvesting antenna 522 can be configured to induce a current in response to being disposed in an alternating electromagnetic field. Additionally, similar to the communications antenna 120, the communications antenna 512 can be configured to communicate data to an external device. The communications antenna 512 can, in some embodiments, include a network interface that is compatible with IEEE 802.15. For example, the communications antenna 512 can be a Bluetooth antenna configured to radiate radiofrequency energy to transmit data to an external device (e.g., to an external antenna such as second external antenna 20 in an external device 11).

The substrate can include multiple layers arranged in a suitable stackup to form a single substrate. For example, as shown in the partial cross-sectional view of FIG. 5B, the substrate can include a series of layers arranged adjacent to one another in series along a thickness dimension of the substrate, between a first broad surface 506a and a second broad surface 506b. As shown in the top plan view of FIG. 5A, the first broad surface 506a and the second broad surface 506b of the substrate can be substantially ovular, such that the flexible circuit is substantially ovular (e.g., racetrack-shaped). It should be understood, however, that the first broad surface 506a and/or the second broad surface 506b of the substrate can be any suitable shape (e.g., circular, polygonal, etc.). The overall substrate can have any suitable thickness between the first broad surface 506a and the second broad surface 506b, such as a thickness that enables the electronics package 108 (or portions thereof) to have sufficient flexibility for conforming to surrounding anatomy when implanted. For example, in some embodiments, the substrate can have a thickness of between about 0.3 mm and about 0.5 mm, or between about 0.40 mm and about 0.42 (e.g., a thickness of about 0.0164 inches, or 0.417 mm).

In some embodiments, as shown in FIG. 5B, the substrate can include at least six layers with conductive material. For example, the flexible circuit 500 includes a substrate with a first layer 510, a second layer 520, a third layer 530, a fourth layer 540, a fifth layer 550, and a sixth layer 560 that can be defined with suitable semiconductor manufacturing processes (e.g., etching). Each of the layers 510, 520, 530, 540, 550, and 560 can include an electrically conductive material (e.g., copper), and may be interspersed with one or more additional layers (e.g., plating copper, polyimide, bonding sheet) and/or interconnected with one or more conductive vias 502. For example, in some embodiments the substrate comprises multiple layers of a heat resistant polymer (such as polyimide) with adhesive material between adjacent layers. The substrate can have one or more vias extending partially or completely through a thickness of the substrate, and one or more electrical connectors can extend through the vias to electrically couple certain electronic components of the electronics package 108, such as the power harvesting antenna 522 and/or the previously discussed electronics component 118.

In some embodiments, the communications antenna 512 is located on a different layer of the multiple layers of the substrate than the power harvesting antenna 522. For example, the communications antenna 512 can be located on the first layer 510, while the power harvesting antenna 522 can be located on one or more layers other than the first layer 510 (e.g., third layer 530 and fourth layer 540, as further described herein). As further described herein, it may be advantageous, in some embodiments, to arrange the communications antenna 512 on a different layer than the power harvesting antenna 522 to increase the physical distance between the two antennas and reduce interference between the two antennas by reducing broadside coupling between the two antennas.

As shown in FIG. 5B, in some embodiments, the first layer 510 can include the communications antenna 512 (e.g., Bluetooth antenna). In some embodiments, the first layer 510 can additionally include circuitry associated with the communications antenna 512, such as a matching circuit 514 and/or a radio chip 516 (e.g., Bluetooth radio chip) as described in further detail below. However, in some embodiments, the matching circuit 514 and/or other portions of the circuitry associated with the communications antenna can be located on other suitable layer(s) of the substrate (e.g., the second layer 520 adjacent to the first layer 510).

In some embodiments, the layer including the communications antenna 512 can be located closer to the first broad surface 506a (upper face of the substrate in the orientation shown in FIG. 5B) than the second broad surface 506b (lower face of the substrate in the orientation shown in FIG. 5B). As such, the communications antenna 512 can have closer proximity to one outer broad surface of the substrate than its opposing broad surface. In some embodiments, the electronics package 108 can be implanted in a patient such that in a typical use case scenario of the neuromodulation device 100, the first broad surface 506a (and hence the communications antenna 512) is positioned closer to the second external antenna 20 (e.g., in the external device 11 such as a mat). This provides a more direct and shorter path between the implanted communications antenna 512 and the second external antenna 20, thereby enabling a stronger and more stable connection between the two antennas.

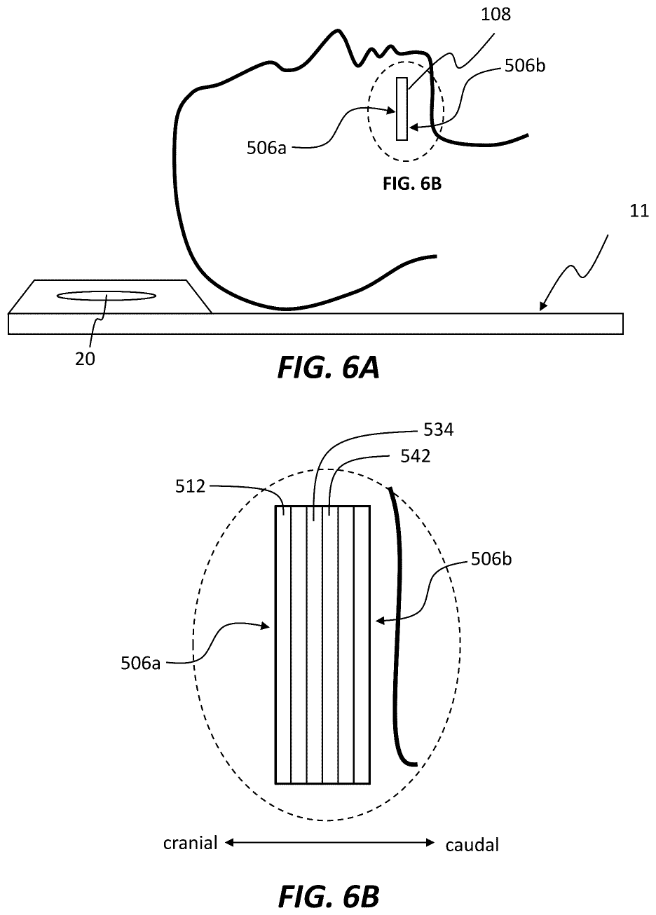

For example, FIG. 6A is a schematic illustration of an electronics package 108 of a neuromodulation device 100, where the electronics package 108 includes a substrate having a first broad surface 506a and a second broad surface 506b located opposite the first broad surface 506a along a thickness direction. The electronics package 108 is configured to be implanted in an under-chin region of the patient in accordance with the implantation process described herein with respect to FIGS. 3A-3F. For example, the electronics package 108 can be configured to be positioned within an under-chin region of the body of the patient with the first broad surface 506a proximate to a mylohyoid muscle of the patient and the second broad surface 506b spaced apart from the mylohyoid muscle by the thickness of the substrate. In other words, the electronics package 108 can be implanted such that the first broad surface 506a is more cranial than the second broad surface 506b. FIG. 6B is a detailed partial view of the electronics package 108 shown in FIG. 6A, including a substrate with a first layer including the communications antenna 512, a third layer including a first coil 534 (also referred to herein as an upper coil) of the power harvesting antenna 522, and a fourth layer including a second coil 542 (also referred to herein as a lower coil) of the power harvesting antenna 522. The communications antenna 512 is located closer to the first broad surface 506a than the second broad surface 506b.

During typical operation of the neuromodulation device, the patient is lying on an external device 11 as shown in FIG. 6A, with the external device 11 positioned proximate a top of the head of the patient. Since the electronics package 108 is implanted such that the first broad surface 506a is located more cranial and closer to the second external antenna 20 in the external device 11, the communications antenna 512 is also located closer to the second external antenna 20. This enables a more direct and shorter path between the communications antenna 512 and the second external antenna 20, which reduces the amount of attenuation of the signal from the communications antenna 512. For example, the signal from the communications antenna 512 is less likely to travel in an indirect or circuitous route before reaching the second external antenna 20 (e.g., around the room in which the patient lies, reflecting off walls, etc.), which otherwise would undesirably attenuate the signal from the communications antenna 512.

The second layer 520, the third layer 530, the fourth layer 540, the fifth layer 550, and the sixth layer 560 can be associated with the power harvesting antenna 522 and/or its corresponding circuitry features. For example, in some embodiments, the third layer 530 and the fourth layer 540 can include corresponding upper and lower coils of the power harvesting antenna 522 as further described in detail below. The second layer 520 (located adjacent to the upper coil of the power harvesting antenna, between the first layer 510 and the third layer 530) and/or the fifth layer 550 (located adjacent to the lower coil of the power harvesting antenna, between the fourth layer 540 and the sixth layer 560) can include traces (and/or other conductive paths) and/or circuitry components in electrical communication with the power harvesting antenna 522, such as through via(s) 502. The sixth layer 560 can include additional power harvesting circuitry that can, for example, be configured to perform filtering, signal processing, and/or other suitable functions for converting current induced in the power harvesting antenna 522 to usable power for the neuromodulation device 100.

The substrate can further include a ground plate 532 providing an electrical ground for the electronics package 108. In some embodiments, the ground plate 532 is located on the third layer 530, and is shown in FIG. 5A as a conductive region centrally located within the coils of the power harvesting antenna 522, which is also partially on the third layer 530. In some embodiments, the power harvesting antenna is closely coupled to the ground plate 532 through a capacitor, such as a 2.2 μF capacitor, such that the power harvesting antenna functions as a ground structure. The ground plate 532 can advantageously be separated from the communications antenna 512 (e.g., on different layers of the substrate) so as to reduce eddy current coupling to the communications antenna 512, which has been found to improve radiation efficiency of the communications antenna 512 and its signal quality. For example, in some embodiments the communications antenna 512 and the ground plate 532 can be on different layers that are spaced apart by at least one intervening layer (e.g., the communications antenna 512 is on the first layer 510 and the ground plate 532 is on the third layer 530, with the second layer 520 intervening between the first layer 510 and the third layer 530). In some embodiments, the communications antenna 512 and the ground plate 532 can be spaced apart (e.g., vertically spaced apart on different layers) by at least about 0.75 mm.

In some embodiments, the flexible circuit 500 can be encapsulated with a suitable coating material configured to encase and/or support the flexible circuit 500. The coating can include a biocompatible material such as, but not limited to, epoxy, urethane, silicone, or other biocompatible materials. In some embodiments, the coating can include multiple layers of distinct materials. In some embodiments, the thickness of the coating is configured to stabilize the impedance of the power harvesting antenna 522 and/or the communications antenna 512. This helps control one factor (RF characteristics of the encapsulation material) that may contribute to attenuation of the signal from the communications antenna 512 as a result of mismatched impedances between the communications antenna 512 and the radio chip 516, as further described below. Furthermore, the coating can be configured to avoid significant degradation of return loss of the communications antenna 512 (e.g., maintain at least a −10 dB of return loss of the communications antenna 512). In some embodiments, the coating includes epoxy material and has a thickness ranging between about 1.8 mm and about 2.2 mm, or a thickness of about 2 mm. The coating can be applied in a uniform manner around flexible circuit 500 so as to have a consistent thickness around the flexible circuit 500, thereby helping to ensure that the loading effect on the communications antenna 512 and/or the performance of the matching circuit 514 (described in further detail below) are more predictable and/or uniform.

### 2. Power Harvesting Antenna

As described elsewhere herein, the first external antenna 12 can be configured to emit an electromagnetic field to induce an electrical current in the power harvesting antenna 522, which can then be supplied to the electronics component 118 and/or conductive elements 114 in the implantable neuromodulation device 100. In some embodiments, the power harvesting antenna 522 can be configured to operate in an RF band, such as at a frequency between 100 kHz and about 900 MHz. In some embodiments, the power harvesting antenna 522 can be configured to operate at a frequency of 6.78 MHz. In some embodiments, the power harvesting antenna 522 comprises a coil or multiple coils. For example, the power harvesting antenna 522 can comprise one or more coils disposed on the flexible substrate.

In some embodiments, the power harvesting antenna 522 comprises multiple coils. For example, the power harvesting antenna 522 can comprise a first coil at one layer of the substrate and a second coil on another layer of the substrate. Ordinarily, this configuration can be susceptible to power losses due to substrate losses and parasitic capacitance between the multiple coils and between the individual coil turns. Substrate losses occur due to eddy currents in the substrate due to the non-zero resistance of the substrate material. Parasitic capacitance occurs when these adjacent components are at different voltages, creating an electric field that results in a stored charge. All circuit elements possess this internal capacitance, which can cause their behavior to depart from that of “ideal” circuit elements.

To reduce such power losses, in some embodiments the power harvesting antenna 522 comprises a two-layer, pancake style coil configuration in which the upper and lower coils are advantageously configured in parallel. As a result, the coils can generate an equal or substantially equal induced voltage potential when subjected to an electromagnetic field. This can help to equalize the voltage of the coils during use, and has been shown to significantly reduce the parasitic capacitance of the power harvesting antenna 522. In this parallel coil configuration, the upper and lower coils are shorted together within each turn. This design has been found to retain the benefit of lower series resistance in a two-coil design while, at the same time, greatly reducing the parasitic capacitance and producing a high maximum power output. Additional details regarding the two-coil configuration can be found in U.S. application Ser. No. 16/866,523, filed May 4, 2020, and U.S. Provisional Application No. 63/573,726, filed Apr. 3, 2024, each of which is incorporated by reference herein in its entirety. In some embodiments, with reference to FIG. 5B, the power harvesting antenna 522 can include a first (upper) coil on the third layer 530 and a second (lower) coil on the fourth layer 540 of the substrate. The upper and lower coils can be shorted together within each coil turn such as with vias 502, in a parallel coil configuration as described above.

As described above, the sixth layer 560 of the substrate can include power harvesting circuitry associated with the power harvesting antenna 522. The power harvesting circuitry can, for example, include components that optimize power efficiency of the power harvesting antenna 522 at the system operating frequency (e.g., 6.78 MHZ). For example, it can be advantageous for power harvesting circuitry to shift and/or otherwise tune a resonance curve of the power harvesting antenna such that across a range of circuit loading scenarios, the system operating frequency is biased toward a lower frequency region of the resonance curve where the power harvesting antenna 522 is less impacted by variations in circuit loading. This tuning of the resonance curve relative to the operating frequency can thus improve power efficiency of the power harvesting antenna 522.

Furthermore, in some embodiments as shown in FIG. 5A, the sixth layer 560 can further include a power harvesting circuitry connection 562 (e.g., electrically conductive path, such as a wire or trace) that connects the outermost turn of the power harvesting antenna 522 to the power harvesting circuitry. The power harvesting circuitry connection 562 can extend orthogonally to a superimposed portion of the communications antenna 512 located on the first layer 530 (that is, the portion of the communications antenna 512 that overlaps with the power harvesting circuitry connection 562). This perpendicular arrangement has been found to further reduce coupling between the power harvesting antenna 522 and the communications antenna 512 and improve signal quality from the communications antenna 512, despite the relative close proximity of the communications antenna 512 to the power harvesting antenna 522 compared to the antenna arrangements in conventional implantable devices.

### 3. Communications Antenna and Matching Circuit

The communications antenna 512 functions to communicate via one or more wireless technologies with an external antenna, such as the external antenna 20 in an external device 11. In some embodiments, the communications antenna 512 is a Bluetooth dipole antenna that is operable with a radio chip 516 (e.g., Bluetooth radio chip) configured for 2-way radio communication. For example, the communications antenna 512 can be configured to dynamically operate within an operating frequency range of between about 2.4 GHz and about 2.483 GHZ.

As described previously herein, due to the close proximity of the communications antenna 512 and the power harvesting antenna 522 located on the same substrate, one significant challenge is the increased risk of interference from the power harvesting antenna 522 in the communications signal from the communications antenna 512. Furthermore, the communications signal from the communications antenna 120 may be attenuated to different degrees as it travels through each material and across each interface between distinct materials, thereby reducing signal quality and/or efficiency of communications. One or more various features of the electronics package 108 can reduce such interference and enable the communications antenna 512 to operate in a suitable manner despite being on the same substrate as the power harvesting antenna 522, and also further address other signal attenuation issues.

For example, in some embodiments, the communications antenna 512 can be radially separated from the power harvesting antenna 522 by a suitable radial separation distance. As shown in FIG. 5A, the communications antenna 512 can be located radially within the innermost coil turn of the power harvesting antenna 522. The communications antenna 512 can be radially separated from the innermost coil turn of the power harvesting antenna 522 by a suitable radial distance to help reduce eddy current induction via broadside coupling. For example, in some embodiments the communications antenna 512 can be radially separated from the innermost coil turn of the power harvesting antenna 522 by a separation distance of between about 0.5 mm and about 0.8 mm (e.g., at least about 0.6 mm).

Furthermore, as described above, in some embodiments the coils of the power harvesting antenna 522 are located on a different layer than the communications antenna 512 to provide physical vertical separation between the power harvesting antenna 522 and the communications antenna 512. The physical separation between the power harvesting antenna 522 and the communications antenna 512 (both in radial separation and vertical separation across different substrate layers) reduces eddy current induction in the communications antenna 512 from the power harvesting antenna 522, thereby reducing loss in communications signal quality resulting from such eddy current induction.

Additionally or alternatively, in some embodiments, the orthogonality of certain electrically conductive paths (e.g., traces, wires) of the power harvesting antenna 522 relative to the communications antenna 512 can further help reduce eddy coupling between the two antennas. For example, as described above, the flexible circuit can include a power harvesting circuitry connection 562 (e.g., wire, trace) that extends orthogonally (rather than parallel, for example) relative to a superimposed or overlapping portion of the communications antenna 512 to connect the outermost coil turn of the power harvesting antenna 522 to the power harvesting circuitry. This orthogonal positioning of the power harvesting circuitry connection 562 may help reduce eddy coupling. Additionally or alternatively, the communications antenna 512 can include an electrically conductive path (e.g., wire, trace) extending orthogonally (rather than parallel, for example) relative to the superimposed or underlying coil turns of the power harvesting antenna 522. For example, the communications antenna 512 can include an electrically conductive path (e.g., trace, wire) extending orthogonally across an underlying portion of the power harvesting antenna 522, from the innermost coil turn of the power harvesting antenna 522 to the outermost coil turn (or radially outside the outermost coil turn). This orthogonal positioning of the communications antenna path may help reduce eddy coupling.

As described above, the communications antenna 512 can be operable with a radio chip 516 for wireless communication. For example, the radio chip 516 can be configured to transmit a radiofrequency signal to the communications antenna 512 to cause the communications antenna 512 to radiate radiofrequency energy, and convert radiofrequency energy received by the communications antenna 512 into a communications signal. In some embodiments, the radio chip 516 has a characteristic impedance over its operating frequency, and the presented impedance of the communications antenna 512 is ideally substantially equal to the radio chip's characteristic impedance for maximum power transfer from the communications antenna 512 to the radio chip 516 and minimal signal loss. However, this characteristic impedance may not necessarily be equal to the presented impedance of the communications antenna 512 once the flexible circuit 500 is implanted. This is because the communications antenna 512 impedance varies as a function of the environment in which it is placed, and is dependent on, for example, the substrate material of the flexible circuit 500, the RF characteristics of the encapsulation coating material of the flexible circuit 500, and the RF characteristics of the tissue in which the implant is placed. In other words, the loading on the communications antenna 512 in different environments changes the communications antenna 512 impedance.

As described above, substrate material and the encapsulation coating material can be formed in a controlled manner (e.g., controlled and limited coating thickness), and their impact on the impedance of the communications antenna 512 can be measured and known (e.g., using a vector network analyzer). This helps control some of the factors that may contribute to attenuation of the signal from the communications antenna 512 as a result of mismatched impedances between the communications antenna 512 and the radio chip 516.

To address the variable impact of tissue on the communications antenna 512 impedance, the flexible circuit 500 can include a matching circuit 514 between the communications antenna 512 and the radio chip 516. The function of the matching circuit 514 is to convert the presented impedance of the communications antenna 512 complex to the characteristic impedance of the antenna interface. This reduces or minimizes signal loss at the interface and improves or maximizes power transfer between the antenna 512 and the radio chip 516. Generally, the matching circuit 514 is configured to have a matching circuit impedance that is equal to the difference between the impedance of the communications antenna 512 and the characteristic impedance of the radio chip 516, when the flexible circuit 500 is implanted in tissue.

For example, in some embodiments the communications antenna 512 and the radio chip 516 are a Bluetooth antenna and a Bluetooth transceiver chip, respectively. In these embodiments, the matching circuit 514 is an RF matching circuit employed at the RF interface between the Bluetooth transceiver chip and the Bluetooth antenna. In some embodiments, the Bluetooth transceiver chip has a characteristic impedance of about 50 Ohms over its operating frequency of 2.4 GHz-2.483 GHZ. As such, the matching circuit 514 can have an impedance equal to the difference between the impedance of the Bluetooth antenna complex and the 50 Ohm characteristic impedance of the Bluetooth transceiver chip, when the flexible circuit 500 is implanted in tissue. In some embodiments, the communications antenna 512 is inductive, but shifts lower with an increase in shunt capacitance.

Generally, the matching circuit 514 is further configured to provide a “flat” RF performance with at least a predetermined threshold amount of return loss and with low variation of the return loss across the operating frequency range of the communications antenna 512. The return loss (as a function of frequency) can be advantageously centered (e.g., be greatest) at the center frequency of the operating frequency range, such that if the communications antenna 512 is operating at either the lowest frequency or the highest frequency of the operating frequency range, the return loss is still likely to be sufficient for efficient operation of the communications antenna 512.

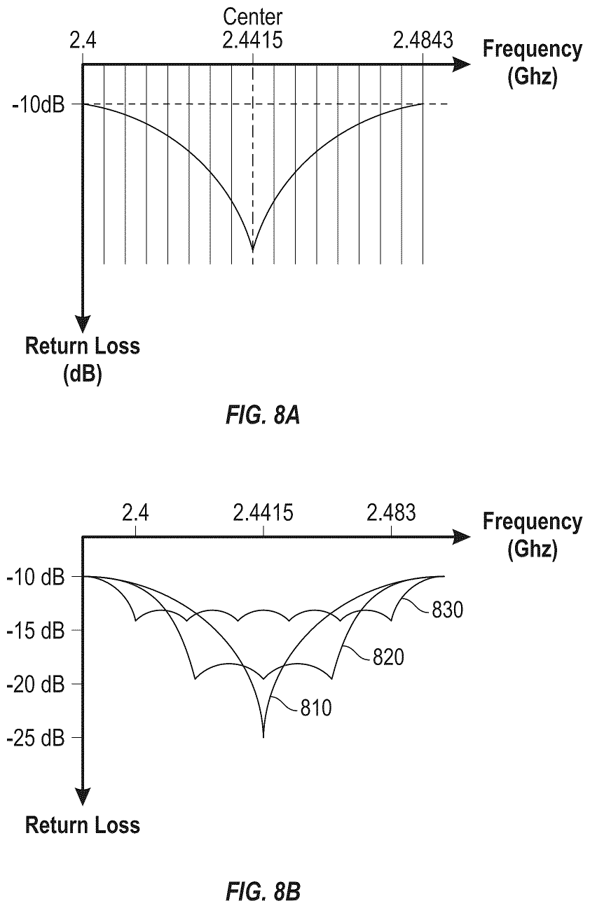

For example, the Bluetooth operating frequency range (2.4 GHz-2.483 GHZ) can be divided into sixteen channels or frequency bands that are approximately equal (e.g., 2.4 GHZ-2.4005 GHZ, 2.4005 GHZ-2.4010 GHz, etc.) and centered around a center frequency of about 2.4415 GHz. FIG. 8A is a plot illustrating return loss vs. operating frequency of an example Bluetooth communications antenna 512. The matching circuit 514 may be configured such that the return loss of the communications antenna 512 within any of channels between the Bluetooth operating range of 2.4 GHz-2.483 GHz is at least about-10 dB, but is centered at the center frequency of 2.4415 GHz in that the return loss is greatest at this center operating frequency. A threshold minimum return loss of −10 dB may be desirable because return loss is on a log scale and the benefits of a threshold value above −10 dB become increasingly marginal.

Additionally, the matching circuit 514 may be configured such that total variation in return loss among the channels is no more than a predetermined threshold, such as about 1 dB. Put another way, the matching circuit 14 may be configured such that total variation in return loss among the channels is no more than about 10% (or no more than about 8%, or no more than about 5%).

The matching circuit 514 can furthermore have a suitable bandwidth for ensuring at least a predetermined threshold amount of return loss (e.g., −10 db) across the desired frequency. For example, a matching circuit with a wider bandwidth can accommodate a sufficiently minimum return loss across a wider operating frequency band, but inherently there may be tradeoffs in the amount of return loss that is possible for any given frequency within the wider operating frequency band. For example, the return loss vs. operating frequency plot of FIG. 8B illustrates the return loss functions 810, 820, and 830 of three matching circuits of varying bandwidth. The return loss function 810 is characteristic of a matching circuit having the narrowest bandwidth, yet greatest maximum return loss, out of the three matching circuits. The return loss function 830 is characteristic of a matching circuit having the widest bandwidth, yet lowest maximum return loss, out of the three matching circuits. The return loss function 820 is characteristic of a matching circuit having a moderate bandwidth and moderate return loss out of the three matching circuits. Accordingly, the matching circuit 514 for any given application can be configured to have a balance of desired bandwidth and return loss, and its characteristics may depend at least in part on the desired operating frequency range of the communications antenna 512. In other words, a wider band matching circuit may be desirable to have a flat performance across a wider operating frequency band.

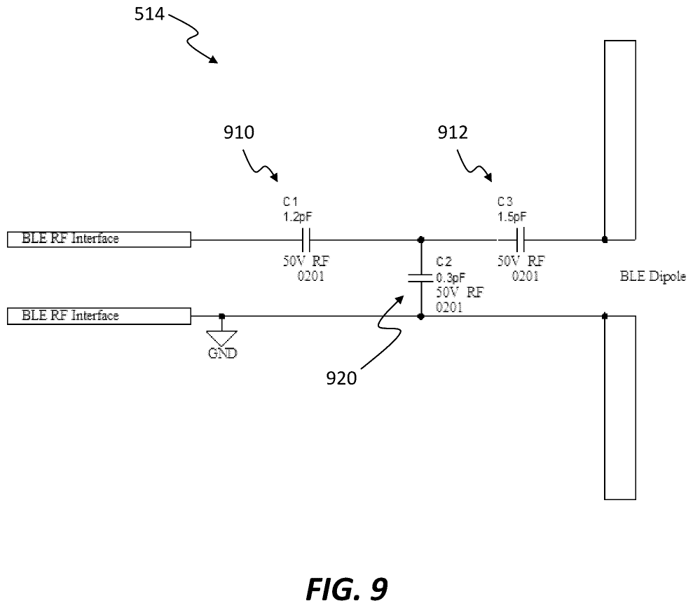

FIG. 9 illustrates a schematic of an example embodiment of a matching circuit 514. The matching circuit 514 is a reactive matching circuit, meaning that the matching circuit 514 does not use resistive components, and includes only reactive elements (e.g., capacitors, inductors). The absence of resistive components (i.e., resistor components aside from conductive features such as conductive traces that have some nominal measure of resistance) helps further reduce power loss, as a significant amount of energy would get lost into any resistive components that are in the matching circuit 514. In some embodiments, the matching circuit 514 can include shunt capacitance, which provides a shunt capacitive shift to tissue that is effective for a variety of tissue scenarios surrounding the implanted electronics package.

The example matching circuit 514 shown in FIG. 9 is a series-shunt-series matching network with three capacitive elements, including two series capacitors 910, 912 and a shunt capacitor 920 located between the series capacitors 910 and 912. Each capacitor has a high self-resonant frequency (SRF) to help ensure that their capacitance value across the Bluetooth operating frequency is consistent and stable. In some embodiments, at least one capacitor has an SRF of at least about 3 GHZ, at least about 3.5 GHZ, at least about 4 GHz, or at least about 4.5 GHz or higher. For example, the series and shunt capacitors 910, 912, and 920 can have an SRF of at least about 4 GHz. In some embodiments, one or more of the capacitors in the matching circuit 514 is a thin-film capacitors, which advantageously have a high SRF compared to multi-layer capacitors. Note that the matching circuit 514 shown in FIG. 8 includes capacitors and is intended for use with a communications antenna 512 that is inductive, but in other embodiments the matching circuit 514 can include inductors and can be used in combination with a communications antenna 512 that is capacitive.

Although the matching circuit 514 shown in FIG. 9 has a topology including three reactive elements, it should be understood that the matching circuit 514 can include additional reactive elements to increase the bandwidth of the matching circuit 514. An odd number of elements can help maintain symmetry in the return loss function, which can be centered (e.g., maximized) around a center frequency of the operating frequency range that is located about halfway between the lowest and highest frequencies of the operating frequency range. For a communications antenna 512 that is inductive as presented to the radio chip 516, the matching circuit 512 can include an odd number of alternating series capacitors and shunt capacitors, beginning with a series capacitor as the first reactive element. For example, in some embodiments the matching circuit 514 can include three reactive elements (two series capacitors with one shunt capacitor located therebetween), or five reactive elements (three series capacitors with two shunt capacitors, where each shunt capacitor is interspersed between a pair of series capacitors), or seven reactive elements (four series capacitors with three shunt capacitors, where each shunt capacitors is interspersed between a pair of series capacitors), with an increased number of reactive elements enabling the matching circuit 514 to have a wider bandwidth. For example, again with reference to FIG. 8B, the return loss function 810 may be characteristic of a matching circuit with three reactive components (e.g., similar to that shown in FIG. 9). The return loss function 820 may be characteristic of a matching circuit with five reactive components, and the return loss function 830 may be characteristic of a matching circuit with seven reactive components. In some embodiments, the matching circuit 514 can include more than seven reactive components (e.g., nine, eleven, etc.) to further increase the bandwidth of the matching circuit 514.

In some embodiments, the matching circuit 514 can function as a filter to filter out the operating frequency of the power harvesting antenna, to reduce or prevent the potential coupling of this operating frequency into the communications antenna 512 that would otherwise cause interference with the signal to/from the communications antenna 512 (e.g., via the front end of the communications radio chip 516). For example, in some embodiments, the matching circuit 514 can function to filter out the fundamental frequency 6.78 MHz of the power harvesting antenna, to reduce or prevent potential coupling of the 6.78 MHz signal into the Bluetooth antenna via the front end of the Bluetooth radio chip. To accomplish this, in some embodiments, the matching circuit 514 can include capacitors having small capacitance values (e.g., less than about 2 pF). Reactive impedance (Xc) of the capacitors and/or other reactive components in the matching circuit 514 is given by Equation 1 below:

$$\\ \begin{matrix}{X}_{c} = \frac{1}{2 π f C} & ( 1 )\end{matrix}$$

where f is the frequency of the signal passing through the component and C is the capacitance value of the capacitor. When C is very small, the capacitors have high impedance, thereby blocking relatively low frequencies (e.g., a 6.78 MHz signal, which is significantly lower than the Bluetooth operating frequency range) from passing through the matching circuit 514, thus filtering out the signal from the power harvesting antenna. At higher frequencies, the capacitors become more of a short, thereby allowing the transmission of desired signals in the Bluetooth operating range through the matching circuit 514. Accordingly, with smaller capacitance values, the matching circuit functions as a filter network that limits any 6.78 MHz coupling into the Bluetooth radio input and reduces or eliminates interference caused by the 6.78 MHz signal. In some embodiments, the matching circuit 514 can be configured to have a reactive impedance of at least about 10K Ohms at the operating frequency. For example, in an example embodiment, the matching circuit 514 includes capacitors with a capacitance value of about 2 pF, which at a frequency of 6.78 MHZ have a reactive impedance of about 12K Ohms.

Another feature of the electronics package 108 for reducing interference and/or attenuation of the communications signal to/from the communications antenna 512 is the increased separation of the communications antenna 512 from the main ground plate 532 of the flexible circuit 500. As described herein, this separation helps to isolate the communications antenna 512 from potentially interfering signals such as from the power harvesting antenna.

Additionally or alternatively, the size and/or shape of the communications antenna can be configured to limit the length of the communications antenna and/or its proximity to the ground plate 532 of the flexible circuit 500 (and/or any other functional ground plates, such as potentially the outermost coil of the power harvesting antenna). In some embodiments, for example, the communications antenna 512 can be a dipole antenna, where the length of each side of the dipole antenna is sized to maintain a suitable separation distance from one or more ground plate(s) in the substrate. In some embodiments, for example, the length of each side of the dipole antenna can be between about 15 mm and about 25 mm, or between about 17 mm and about 23 mm, or about 22 mm long.

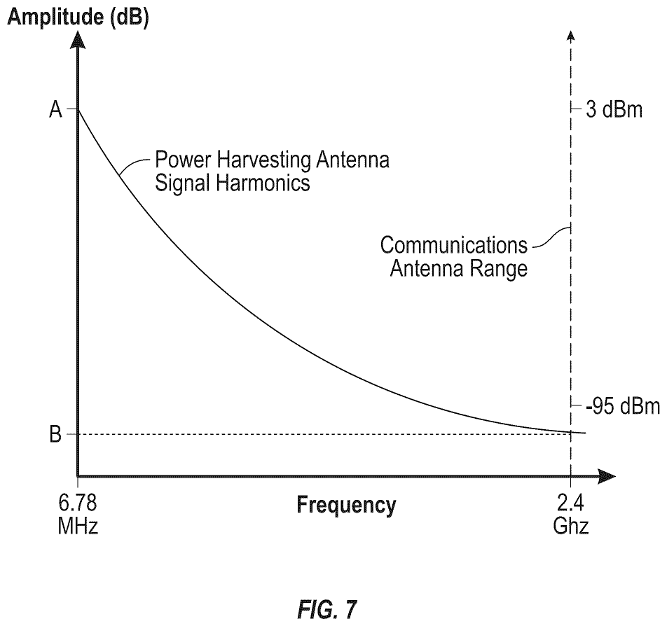

Thus, in some embodiments, various features of the flexible circuit 500 help enable the avoidance of power coupling or inductive coupling that is caused by the power harvesting antenna 522 being in close proximity to the communications antenna 512. For example, in embodiments in which the power harvesting antenna 522 operates at a fundamental frequency of 6.78 MHz and the communications antenna 512 operates within an operating dynamic range of between about 2.4 GHz and about 2.483 GHZ, harmonics of the 6.78 MHz signal can potentially sit within the operating dynamic range of the communications antenna 512, thereby causing interference. As such, in some embodiments, to avoid interference, the flexible circuit 500 is configured (e.g., the power harvesting antenna 522 and the communications antenna 512 are sufficiently physically separated radially and/or vertically across layers) such that the amplitude of harmonics of the power harvesting antenna's 6.78 MHz signal is below floor of the operating dynamic range of the communications antenna at 2.4 GHZ). For example, as illustrated in FIG. 7, in some embodiments the communications antenna has a range between about 3 dBm to about −95 dBm when operating within the Bluetooth operating dynamic range of 2.4 GHz-2.473 GHZ. The power harvesting antenna signal has a max amplitude at the fundamental frequency 6.78 MHz and has an amplitude that decreases at higher frequencies, but the harmonics of the power harvesting antennal signal can ordinarily have sufficient signal strength to be greater than −95 dBm and thus interfere with the Bluetooth communications signal from the communications antenna 512. However, as illustrated in FIG. 7, the flexible circuit 500 is configured such that harmonics of the power harvesting antenna signal has an amplitude below −95 dBm and hence below the operating dynamic range of the communications antenna 512. Various features described herein help enable this performance and result in reduced interference from the power harvesting antenna 522, including (i) sufficient radial and vertical separation between the power harvesting antenna 522 and the communications antenna 512, (ii) orthogonality of certain electrically conductive paths (e.g., traces, wires) of the power harvesting antenna relative to the communications antenna (e.g., power harvesting circuitry path 562), (iii) the nature of the matching circuit 514, (iv) separation of the communications antenna 512 from the ground plane 532, and/or (v) limited size of the communications antenna 512. In other words, some or all of these technical solutions help enable strong enough broadcasting of the communications signal from the communications antenna 512 despite the close proximity of the communications antenna to the power harvesting antenna 522 and the presence of the large RF field associated with the power harvesting antenna 522.

### 4. Other Examples of Electronics Packages

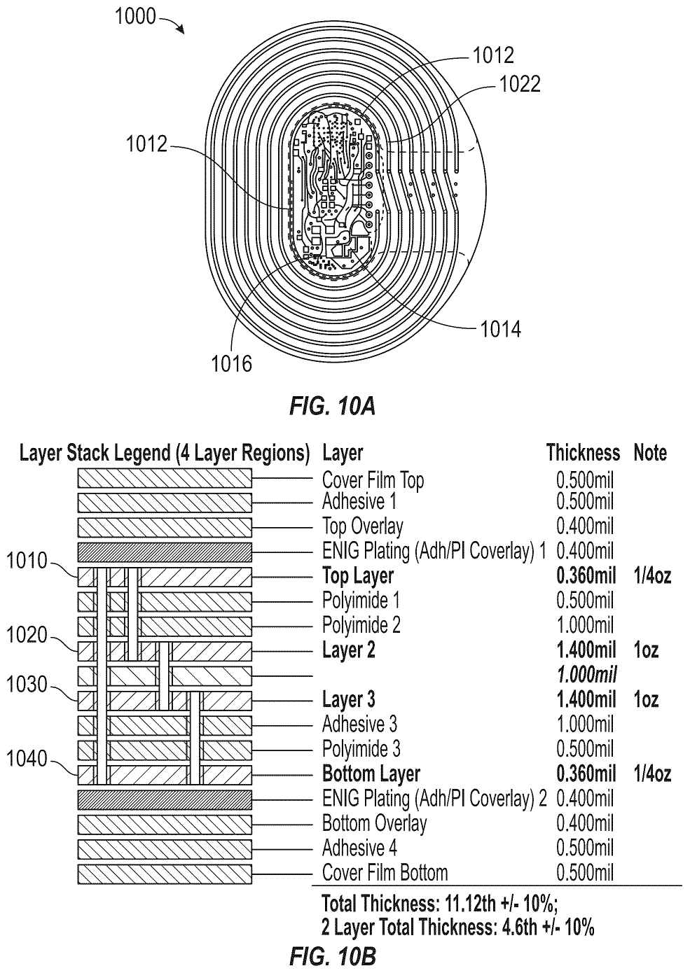

Other examples of electronics packages are shown in FIGS. 10A-13B.

FIGS. 10A and 10B illustrate an example embodiment of an electronics package including a flexible circuit 1000. The flexible circuit 1000 includes four layers in a central region including a power harvesting antenna 1022 (e.g., similar to the power harvesting antenna 522), a communications antenna 1012 (e.g., similar to the communications antenna 512) such as a Bluetooth dipole antenna, a communications radio chip 1016 (e.g., Bluetooth radio chip) and a matching circuit 1014 configured to convert the presented communications antenna complex impedance (e.g., Bluetooth antenna complex impedance) to a required impedance of the antenna interface. Like the matching circuit 514 described herein, the matching circuit 1014 is configured to minimize or reduce signal loss at the antenna interface and help ensure increased or maximum power transfer from the communications antenna 1012 to the communications radio chip 1016. The perimeter or outskirts of the flexible circuit around this central region can be thinner and/or include fewer layers, such as only two layers (e.g., including insulation and/or encapsulation material). The thin dimension and/or fewer layers of the perimeter of the flexible circuit can help enable the flexible circuit 1000 to be more flexible around its edge portions, which may allow the flexible circuit 1000 to conform more easily to surrounding anatomy when implanted in a patient.

FIG. 10B illustrates the stack of layers in the central region of the flexible circuit 1000. A first layer 1010 in the flexible circuit 1000 includes the communications antenna 1012, the communications radio chip 1016, and the matching circuit 1014. A second layer 1020 and a third layer 1030 can include upper and lower coils, respectively, of the power harvesting antenna 1022 (which can be similar to power harvesting antenna 522 described herein). A fourth layer 1040 can include power harvesting circuitry and/or other electronics. Like the flexible circuit 500, the flexible circuit 1000 can be encapsulated in a coating material. The flexible circuit 1000 can further be coupled to a lead body of a neuromodulation device 100.

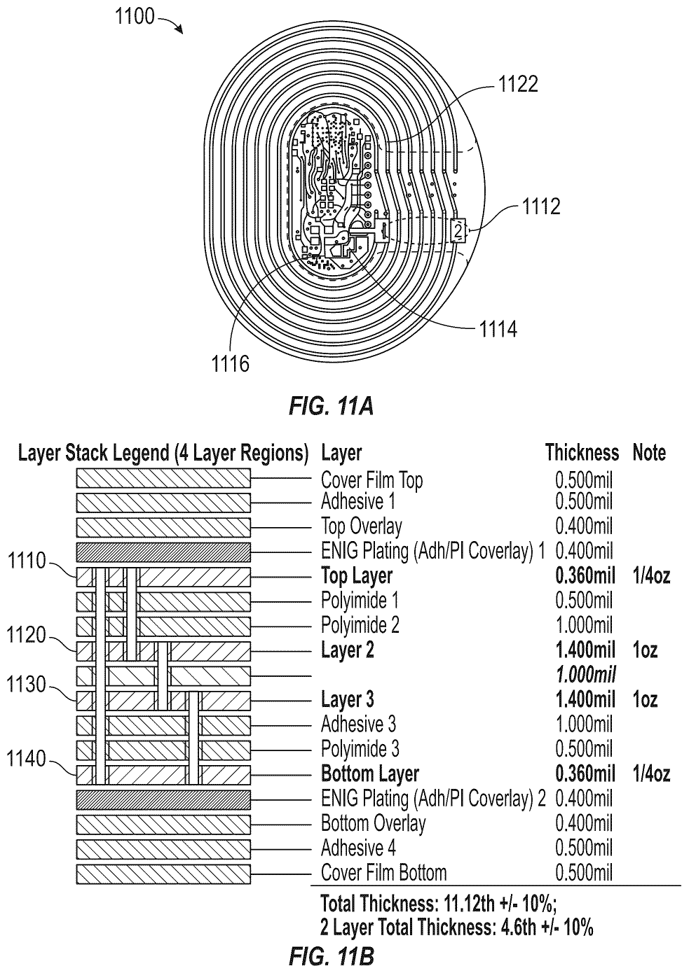

FIGS. 11A and 11B illustrate an example embodiment of an electronics package including a flexible circuit 1100. Generally, the flexible circuit 1100 is similar to the flexible circuit 1000 except as described herein. For example, the flexible circuit 1100 includes four layers in a central region including a power harvesting antenna 1122 (e.g., similar to the power harvesting antenna 522), and a communications radio chip 1116 (e.g., Bluetooth radio chip). However, instead of a Bluetooth dipole antenna, the flexible circuit 1100 includes a commercially, off-the-shelf Bluetooth “chip” antenna, which has the advantage of a compact design without loose wires and the benefit of a well-tested antenna. The flexible circuit 1100 also includes a matching circuit 1114 configured to convert the presented communications antenna complex impedance (e.g., Bluetooth antenna complex impedance) to a required impedance of the antenna interface. Like the matching circuit 514 described herein, the matching circuit 1114 is configured to minimize or reduce signal loss at the antenna interface and help ensure increased or maximum power transfer from the communications antenna 1112 to the communications radio chip 1116. Like the flexible circuit 1000 described above with reference to FIG. 10A, the perimeter or outskirts of the flexible circuit around this central region can be thinner and/or include fewer layers, such as two layers.

FIG. 11B illustrates the stack of layers in the central region of the flexible circuit 1100. A first layer 1110 in the flexible circuit 1100 includes the communications antenna 1112, the communications radio chip 1116, and the matching circuit 1114. A second layer 1120 and a third layer 1130 can include upper and lower coils, respectively, of the power harvesting antenna 1122 (which can be similar to power harvesting antenna 522 described herein). The power harvesting antenna 1122 can be closely coupled to a ground plate by a capacitor (e.g., 2.2 μF capacitor) such that the power transmitter antenna coils act as a ground structure. A fourth layer 1140 can include power harvesting circuitry and/or other electronics. Like the flexible circuit 500, the flexible circuit 1100 can be encapsulated in a coating material. The flexible circuit 1100 can further be coupled to a lead body of a neuromodulation device 100.

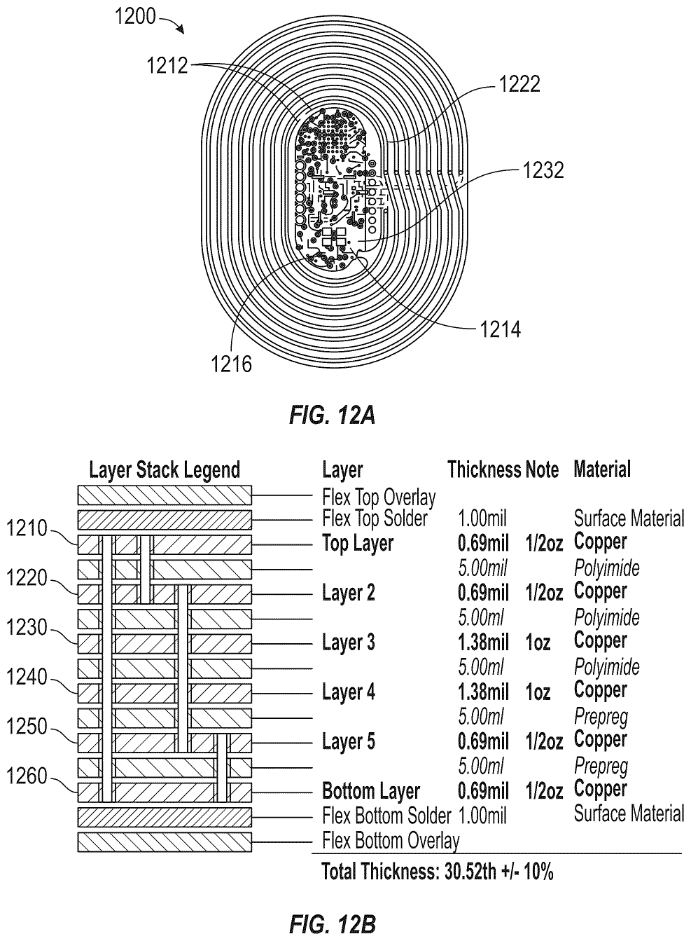

FIGS. 12A and 12B illustrate an example embodiment of an electronics package including a flexible circuit 1200. Generally, the flexible circuit 1200 is similar to the flexible circuit 500 except as described herein. Like the flexible circuit 500, the flexible circuit 1200 includes six layers on which a power harvesting antenna 1222 (e.g., similar to the power harvesting antenna 522), a communications antenna 1212 (e.g., similar to the communications antenna 512) such as a Bluetooth dipole antenna, a communications radio chip 1216 (e.g., Bluetooth radio chip) and a matching circuit 1214 configured to convert the presented communications antenna complex impedance (e.g., Bluetooth antenna complex impedance) to a required impedance of the antenna interface. The matching circuit 1214 is configured to minimize or reduce signal loss at the antenna interface and help ensure increased or maximum power transfer from the communications antenna 1212 to the communications radio chip 1216.

FIG. 12B illustrates the stack of layers in the flexible circuit 1200. A first layer 1210 in the flexible circuit 1200 includes the communications antenna 1212 formed as a dipole from a PCB trace and/or other electrically conductive path, the communications radio chip 1216, and the matching circuit 1214. A third layer 1230 and a fourth layer 1240 can include upper and lower coils, respectively, of the power harvesting antenna 1222 (which can be similar to power harvesting antenna 522 described herein). The third layer 1230 further includes a ground plane 1232 (shown in FIG. 12A) to which the power harvesting antenna 1222 is coupled by a 2.2 μF capacitor, such that the power harvesting antenna 1222 functions as a ground structure. A second layer 1220 and a fifth layer 1250, located adjacent to the power harvesting antenna's upper and lower coils, respectively, can include traces and/or other electrically conductive path(s) associated with the power harvesting antenna, and a sixth layer 1260 can include power harvesting circuitry and/or other electronics. Like the flexible circuit 500, the flexible circuit 1200 can be encapsulated in a coating material. The flexible circuit 1200 can further be coupled to a lead body of a neuromodulation device 100.

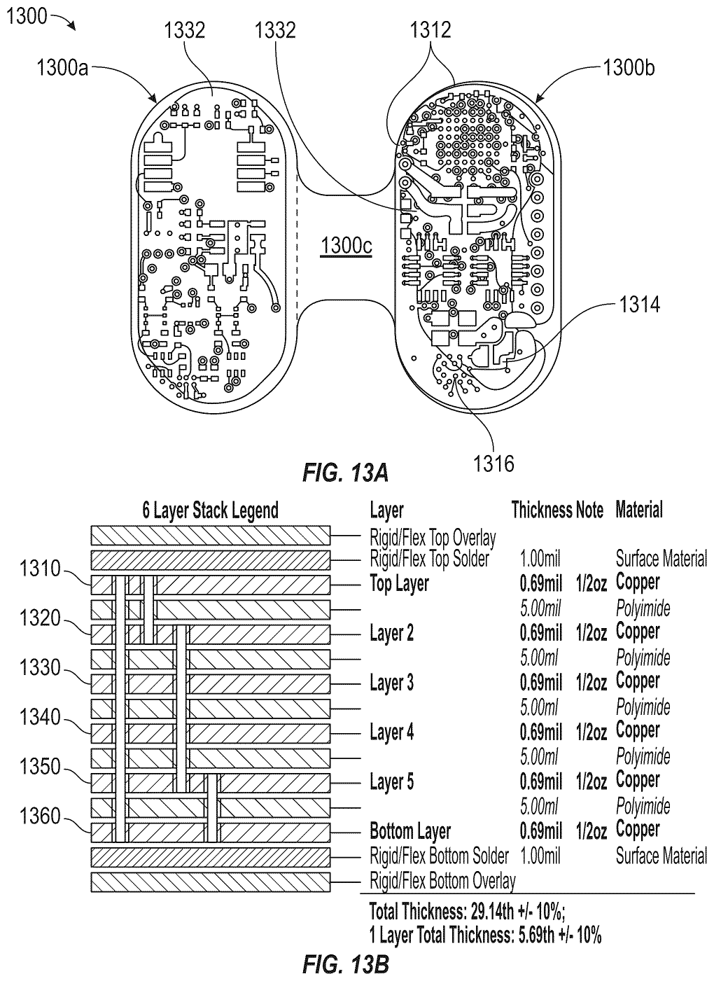

FIGS. 13A and 13B illustrate an example embodiment of an electronics package including a flexible circuit 1300. Generally, the flexible circuit 1300 is similar to the flexible circuit 500 except as described herein. Like the flexible circuit 500, the flexible circuit 1300 includes six layers on which a power harvesting antenna (not shown) (e.g., similar to the power harvesting antenna 522), a communications antenna 1312 (e.g., similar to the communications antenna 512) such as a Bluetooth dipole antenna, a communications radio chip 1316 (e.g., Bluetooth radio chip) and a matching circuit 1314 configured to convert the presented communications antenna complex impedance (e.g., Bluetooth antenna complex impedance) to a required impedance of the antenna interface. The matching circuit 1314 is configured to minimize or reduce signal loss at the antenna interface and help ensure increased or maximum power transfer from the communications antenna 1312 to the communications radio chip 1316. However, in flexible circuit 1300, the electronics are split into two regions 1300a and 1300b that are connected by a flexible or foldable bridge 1300c. The power harvesting antenna is located on a second PCB (not shown).

FIG. 13B illustrates the stack of layers in the flexible circuit 1300. A first layer 1310 in the flexible circuit 1300 includes the communications antenna 1312 formed as a dipole from a PCB trace and/or other electrically conductive path, the communications radio chip 1316, and the matching circuit 1314. As described above, the electronics are split into two regions 1300a and 1300b, in order to increase the separation distance between the power transmitting antenna and the communications dipole antenna 1312, and to keep more of the electronics and the ground plane 1332 farther away from the communications dipole antenna 1312. Upper and lower coils of the power harvesting antenna are formed on a separate PCB (not shown) that is electrically and mechanically attached to the PCB shown in FIG. 13A. Furthermore, the PCB shown in FIG. 13A is folded over by folding the bridge member 1300c to form a sandwich, and then mechanically secured together and with the power transmitting antenna PCB. Like the flexible circuit 500, the flexible circuit 1300 can be encapsulated in a coating material. The flexible circuit 1300 can further be coupled to a lead body of a neuromodulation device 100.

### 5. Other Examples of Communication Antenna Arrangements

In some embodiments, at least a portion of the electronics package 108 can be included in the lead body of the neuromodulation device 100. For example, the communications antenna 120 can be located at least partially in the extension portion 106 and/or arms 122, 124 of the lead body 104. This can, for example, help increase physical separation between the power harvesting antenna 116 (or 522) in the electronics package 108 and the communications antenna 120, thereby reducing interference as a result of eddy current coupling between the two antennas.

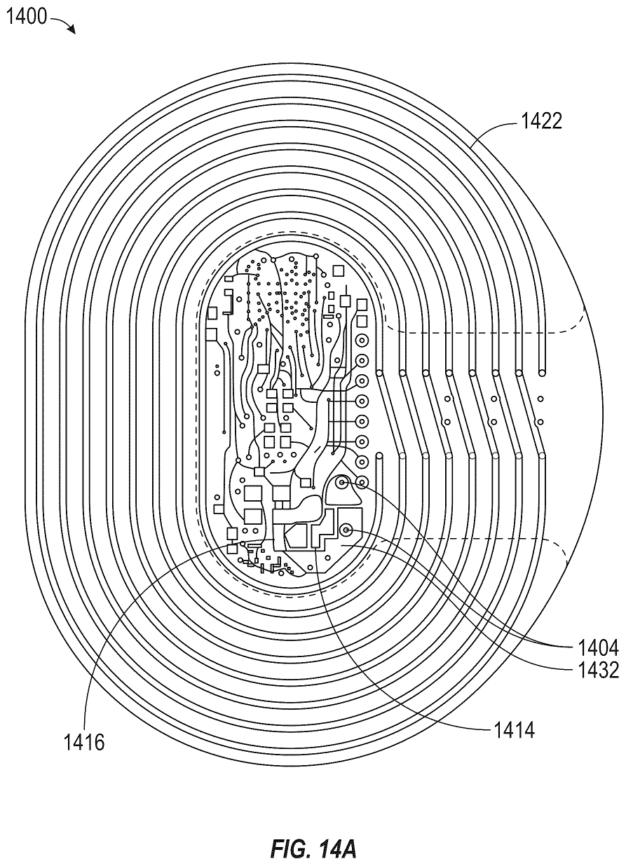

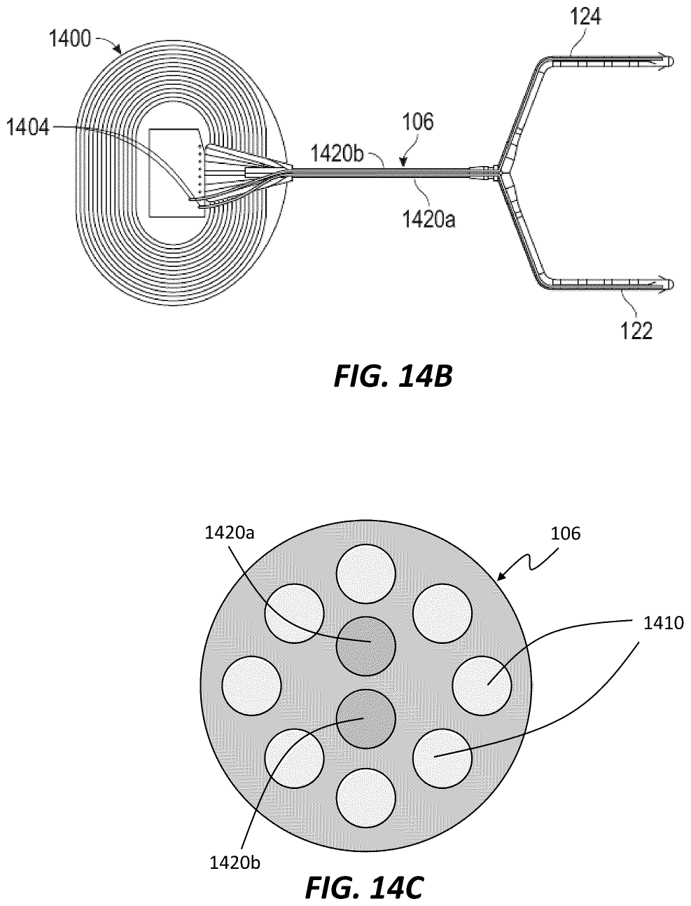

For example, as shown in FIGS. 14A-14C, in some embodiments, the communications antenna 120 can be a dipole antenna with two wires that extend along the extension portion 106 of the lead body. FIG. 14A illustrates an example flexible circuit 1400 that is similar to the flexible circuit 500 except as described herein. For example, like the flexible circuit 500, the flexible circuit 1400 can include a power harvesting antenna 1422 (e.g., including an upper coil and a lower coil on different layers of the flexible circuit substrate), a ground plate 1432 to which the power harvesting antenna 1422 is closely coupled (e.g., by a 2.2 μF capacitor) such that the power harvesting antenna 1422 act as a ground structure, a communications radio chip 1416 (e.g., Bluetooth radio chip), and a matching circuit 1414 configured to convert the presented communications antenna complex impedance (e.g., Bluetooth antenna complex impedance) to a required impedance of the antenna interface. Like the matching circuit 514 described herein, the matching circuit 1414 is configured to minimize or reduce signal loss at the antenna interface and help ensure increased or maximum power transfer from the communications antenna 120 to the communications radio chip 1416. The flexible circuit 1400 further includes two plated through holes 1404 to which the ends of two wires (not shown) of the communications antenna 120 are soldered.

As shown in FIG. 14B, the two communications antenna wires 1420a, 1420b can extend along the extension portion 106 of the lead body toward the arms 122, 124 of the lead body. The antenna wires can, for example, be co-extruded with the shaft of the extension portion 106 along with conductive wires 1410 coupled to the conductive elements 114 (e.g., electrodes), or alternatively can be inserted through respective lumens of the extension portion 106. The first antenna wire 1420a can continue along to one arm of the lead (e.g., arm 122 of the lead body), while the second antenna wire 1420b can continue along to another arm of the lead (e.g., arm 124 of the lead body). Polymeric material of the extension portion 106 and/or the arms 122, 124 of the lead body can insulate the antenna wires 1420a, 1420b.

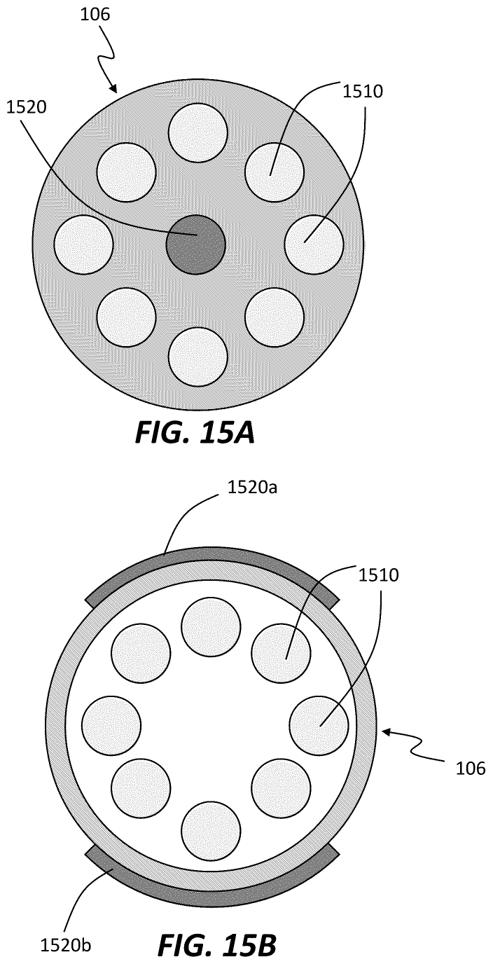

As another example, in some embodiments, as shown in FIG. 15A, the communications antenna can include a single antenna wire 1520 that extends along the extension portion 106 of the lead body. One end of antenna wire 1520 can be soldered to a flexible circuit (not shown) similar to the antenna wires 1420a, 1420b described above with reference to FIGS. 14A-14C, while the other end of the antenna wire 1520 can extend toward the arms 122, 124 of the lead body along the extension portion 106. The antenna wire 1520 can, for example, be co-extruded with the shaft of the extension portion 106 along with conductive wires 1510 coupled to the conductive elements 115 (e.g., electrodes), or alternatively the antenna wire 1520 can be inserted in a lumen of the extension portion 106. The antenna wire 1520 can split and branch into separate wires near the lead bifurcation, and the separate wires can continue along respective arms 122, 124 of the lead. Like the example described above with respect to FIGS. 14A-14C, polymeric material of the extension portion 106 and/or the arms 122, 124 of the lead body can insulate the antenna wire 1520 and/or antenna wires

As another example, in some embodiments, as shown in FIG. 15B, conductive trace(s) and/or other conductive path(s) for the communications antenna 120 can be arranged on an outer surface of the extension portion 106 of the lead body. For example, a first trace 1520a and a second trace 1520b can be plated, printed, or otherwise disposed on the outer sidewall of the extension portion 106 and extend along the extension portion 106 between a flexible circuit (e.g., electronics package 108) and the arms 122, 124 of the lead body. The first trace 1520a can continue to extend along one arm (e.g., arm 122) of the lead body, while the second trace 1520b can continue to extend along another arm (e.g., arm 124) of the lead body. While the traces 1520a, 1520b are shown opposite one another across the cross-section of the extension portion 106, they may be circumferentially positioned relative to each other in any suitable manner (e.g., unequally circumferentially distributed). Furthermore, although two traces are shown in FIG. 15B, it should be understood that in some embodiments, a single trace can be plated, printed, or otherwise disposed on the outer sidewall of the extension portion 106, then branch into separate traces that extend along respective arms 122, 124 of the lead body.

### CONCLUSION

Although many of the embodiments are described above with respect to systems, devices, and methods for modulation of a hypoglossal nerve of a patient, the technology is applicable to other applications and/or other approaches, such as modulation of other nerves of a patient. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-26E.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An implantable device comprising:
a flexible circuit including a substrate carrying:
a power harvesting antenna configured to operate at a first frequency, the power harvesting antenna being configured to induce a current in response to being disposed in an alternating electromagnetic field; and
a Bluetooth antenna configured to operate at a second frequency different than the first frequency, the Bluetooth antenna being configured to radiate radiofrequency energy to transmit data to an external device.

2. The device of claim 1, wherein the power harvesting antenna is configured to operate between about 100 kHz and about 900 MHz and the Bluetooth antenna is configured to operate between about 2.4 GHz and about 2.483 GHz.

3. The device of claim 1, wherein the substrate comprises a first broad surface and a second broad surface opposite the first broad surface along a thickness of the substrate.

4. The device of claim 1, wherein each of the first and second broad surfaces is substantially ovular.

5. The device of claim 1, wherein the substrate comprises multiple layers.

6. The device of claim 5, wherein the Bluetooth antenna is located on a different layer of the multiple layers of the substrate than the power harvesting antenna.

7. The device of claim 5, wherein the power harvesting antenna is located on at least two of the multiple layers of the substrate.

8. The device of claim 1, wherein the power harvesting antenna comprises a plurality of turns and the Bluetooth antenna is located radially within an innermost turn of the plurality of turns.

9. The device of claim 8, wherein the Bluetooth antenna is radially separated from the innermost turn of the power harvesting antenna by at least about 0.5 mm.

10. The device of claim 8, wherein the flexible circuit further comprises power harvesting circuitry and a power harvesting circuitry connection connecting an outermost turn of the plurality of turns to the power harvesting circuitry, wherein the power harvesting circuitry connection extends orthogonally to a superimposed portion of the Bluetooth antenna.

11. The device of claim 8, wherein the Bluetooth antenna comprises a Bluetooth antenna path extending from a first end to a second end, and wherein the first end is radially within an innermost turn of the plurality of turns of the power harvesting antenna and the second end is radially outside of an outermost turn of the plurality of turns of the power harvesting antenna, such that the Bluetooth antenna path extends across the plurality of turns.

12. The device of claim 11, wherein the Bluetooth antenna path extends orthogonally across at least a portion of the plurality of turns of the power harvesting antenna.

13. The device of claim 1, wherein the power harvesting antenna and the Bluetooth antenna are physically separated such that an amplitude of a signal generated by flow of induced current within the power harvesting antenna at the second frequency is less than a noise floor of the Bluetooth antenna.

14. The device of claim 1, wherein the flexible circuit comprises a ground plate electrically coupled to the power harvesting antenna and electrically isolated from the Bluetooth antenna.

15. The device of claim 1, wherein the flexible circuit comprises a matching circuit electrically coupled to the Bluetooth antenna, wherein the matching circuit is configured to prevent or limit transmission of a component of the alternating electromagnetic field at the first frequency through the matching circuit.

16. The device of claim 1, wherein the second frequency is within a predetermined frequency range defined by a maximum frequency and a minimum frequency.

17. The device of claim 16, wherein a return loss of the Bluetooth antenna at any given frequency within the predetermined frequency range is at least −10 dB.

18. The device of claim 16, wherein the predetermined frequency range is discretized into a plurality of frequency bands, and wherein a return loss of the Bluetooth antenna within each frequency band of the plurality of frequency bands is within 10% of the return loss of the Bluetooth antenna within every other frequency band of the plurality of frequency bands.

19. The device of claim 16, wherein a return loss of the Bluetooth antenna is maximized at a center frequency of the predetermined frequency range, the center frequency being about halfway between the minimum frequency and the maximum frequency.

20. The device of claim 1, wherein the Bluetooth antenna is configured to have a first impedance once the implantable device is positioned within a patient, and wherein the flexible circuit comprises:
a Bluetooth module having a second impedance different than the first impedance, the Bluetooth module being configured to transmit a radiofrequency signal to the Bluetooth antenna to cause the Bluetooth antenna to radiate the radiofrequency energy; and
a matching circuit electrically coupled to and positioned between the Bluetooth module and the Bluetooth antenna, the matching circuit having a third impedance, the third impedance being equal to a difference between the second impedance and the first impedance.

21. A system comprising:
an external device configured to be positioned external to a body of a patient, the external device comprising an external Bluetooth antenna; and
an implantable device comprising a flexible circuit including a substrate having a first broad side and a second broad side opposite the first broad side along a thickness of the substrate, the substrate carrying an implantable Bluetooth antenna located closer to the first broad side than the second broad side along the thickness of the substrate, wherein the implantable device is configured to be positioned within the body of the patient such that the first broad side is located closer to the external Bluetooth antenna than the second broad side.

22. A method comprising:
positioning an external device proximate to a body of a patient, the external device comprising an external Bluetooth antenna; and
implanting an implantable device within the body of the patient, the implantable device comprising a flexible circuit including a substrate having a first broad side and a second broad side opposite the first broad side along a thickness of the substrate, the substrate carrying an implantable Bluetooth antenna located closer to the first broad side than the second broad side along the thickness of the substrate,
wherein implanting the implantable device comprises positioning the implantable device within the body of the patient such that the first broad side is located closer to the external Bluetooth antenna than the second broad side.
